(19) United States
(12) Patent Application Publication
(10) Pub. No.: US 2024/0043813 A1
(43) Pub. Date: Feb. 8, 2024

(54) ENTEROVIRUS PURIFICATION WITH CATION EXCHANGE CHROMATOGRAPHY

(71) Applicants: Spyrdion KONSTANTINIDIS, West Point, NJ (US); Murphy POPLYK, West Point, NJ (US); Andrew Ryan SWARTZ, West Point, NJ (US); MERCK SHARP & DOHME LLC, Rahway, NJ (US)

(72) Inventors: Spyridon KONSTANTINIDIS, West Point, PA (US); Murphy POPLYK, West Point, PA (US); Andrew Ryan SWARTZ, West Point, PA (US)

(73) Assignee: MERCK SHARP & DOHME LLC, Rahway, NJ (US)

(21) Appl. No.: 18/257,334

(22) PCT Filed: Dec. 16, 2021
(86) PCT No.: PCT/US2021/063647

§ 371 Date: Jun. 14, 2023

Related U.S. Application Data
(60) Provisional application No. 63/211,162, filed on Jun. 16, 2021.
(60) Provisional application No. 63/126,743, filed on Dec. 17, 2020.

(51) Int. Cl. C12N 7/02; B01D 15/36; B01D 15/20; B01D 15/16

(52) U.S. Cl. CPC C12N 7/02; B01D 15/362; B01D 15/203; B01D 15/166; C12N 2770/32351

(57) ABSTRACT

The present invention relates to a cation exchange chromatography process for the purification of enteroviruses.

### FIELD OF THE INVENTION

The present invention relates to a cation exchange chromatography process for the purification of enteroviruses.

### BACKGROUND OF THE INVENTION

The Enterovirus genus of the Picornaviridae family are small, non-enveloped, single stranded positive sense RNA viruses that contain several species of human pathogens including polioviruses, coxsackieviruses, echoviruses, numbered enteroviruses, and rhinoviruses [1]. Aside from the well-studied poliovirus, there has been an influx of research into the development of vaccines and therapeutics for diseases caused by non-polio enteroviruses such as EV-A71 (hand foot and mouth disease) [2], EV-D68 (respiratory disease) and Coxsackievirus A24 (acute hemorrhagic conjunctivitis) [3]. Enteroviruses have also been evaluated for use as oncolytic viral immunotherapies [4]. Coxsackievirus A21 (CVA21), derived from the wild-type strain, is currently being evaluated in phase 1b/2 clinical trials as a treatment for multiple types of cancer due to its selective infection and oncolysis of tumors overexpressing cell surface receptors ICAM-1[5].

The increasing demand for enterovirus viral vaccines and immunotherapies could challenge the conventional production platform. Gradient ultracentrifugation is commonly employed for the enrichment of full, genome containing capsids and impurity clearance, but may be a potential bottleneck in the purification process due to its low-throughput and labor-intensive protocols [6]. As evidenced by the recombinant adeno-associated viral gene therapy purification platform, a shift from gradient ultracentrifugation towards chromatography-based methods may improve scalability and productivity [7]. No chromatographic technique has been demonstrated for empty (lacking genome; product impurity) and full (genome containing; target product) enterovirus particle separation. There remains a need for a chromatography-based alternative to gradient ultracentrifugation that is capable of removing empty capsids and contaminating impurities to produce a purified composition of infectious, mature virions. This would enable an enterovirus purification process that is more suitable for large-scale commercial manufacturing.

### SUMMARY OF THE INVENTION

The present invention comprises use of cation exchange chromatography to purify enterovirus from one or more impurities. In another aspect, the present invention provides use of glutathione affinity chromatography prior to the cation exchange purification. In one embodiment, the method selectively captures and enriches genome-containing full mature enterovirus virions from infected host-cell culture harvests, thereby removing one or more impurities such as non-infectious genome-lacking enterovirus procapsids, host-cell proteins (HCPs), host-cell DNA (HC-DNA), and media-related impurities such as bovine serum albumin (BSA).

### BRIEF DESCRIPTION OF THE DRAWINGS

The foregoing and other objects, features and advantages of the invention will be apparent from the following more particular description of various embodiments of the invention, as illustrated in the accompanying drawings.

FIG. 1: Enterovirus morphogenesis and assembly. Five protomers consisting of VP0+VP1+VP3 assemble to form a pentamer. Empty procapsids may be formed from the reversible assembly of free pentamers. After 12 pentamers condense and encapsidate the newly synthesized genome on a replication organelle to form a provirion, VP0 is autocatalytically cleaved to form VP4+VP2 and a mature virion is formed. Mature virions are the only particle containing VP4 and are capable of being infectious, but not all mature virions may be infectious. Mature virions may degrade into A-particles and empty capsids of A-particles. Adapted from [8].

FIG. 2A-F: Poros™ 50 HS chromatography traces obtained by plotting, on the left Y-axis, the measured absorbance of the collected fractions at 260 nm, and normalizing it over the difference between the optical absorbances at 990 nm and 900 nm, against the number of the collected fractions, or CVs, at conditions: (A) pH 3.8 and 0.45 M NaCl; (B) pH 4.0 and 0.45 M NaCl; (C) pH 4.2 and 0.3 M NaCl; (D) pH 4.5 and 0.05 M NaCl; (E) pH 5.0 and 0.05 M NaCl; (F) pH 6.0 and 0.05 M NaCl. In each of (A)-(F) solid lines with open square (□) and open circle (∘) markers denote duplicated measurements whereas the dashed line (-) denotes the salt concentration per fraction (right Y-axis). The x-axis is offset to start at the last fraction of the load for each separation. Results shown in (A)-(F) used material generated through upstream process B.

FIG. 3A-F: Chromatography traces obtained by plotting, on the left Y-axis, the measured absorbance of the collected fractions at 260 nm, and normalizing it over the difference between the optical absorbances at 990 nm and 900 nm, against the number of the collected fractions, or CVs, for: (A) Resin Capto S ImpAct at pH 4.0 and 0.05 M NaCl; (B) Resin Capto SP ImpRes at pH 4.0 and 0.05 M NaCl; (C) Resin Nuvia HR-S at pH 4.0 and 0.05 M NaCl; (D) Resin Nuvia S at pH 4.0 and 0.05 M NaCl; (E) Resin Capto S at pH 4.0 and 0.05 M NaCl; (F) Resin Nuvia HP-Q at pH 9.0 and 0.05 M NaCl. In each of (A)-(E) the solid line with open circle (∘) marker denotes single measurement. In (F) the solid lines with open square (□) and open circle (∘) markers denote duplicated measurements. In each of (A)-(F) the dashed line (-) denotes the salt concentration per fraction (right Y-axis). In (F) the x-axis is offset to start at the last fraction of the load. Results shown in (A)-(F) used material generated through upstream process B.

FIG. 4A-G: SDS-PAGE gels of affinity chromatography elution product (Feed), its 3-fold dilution in concentrated binding buffer (Load), and of the Poros™ 50 HS elution pool (E3) and strip pool (S). The content of each lane per gel is shown in G. For Gels (A)-(C) lanes 6-9 are duplicates of lanes 2-5. Gels (D)-(F) contain two conditions per gel and their duplicated samples are spread across gels. For example, Lanes 2-6 in gel (D) are duplicated in gel (E) and lanes 6-9 respectively. The contents of each lane per gel are shown in (G). Band VP0 is characteristic of empty procapsids alone whereas band VP2 is characteristic of full mature virus particles alone. Results shown in (A)-(G) used material generated through upstream process B.

FIG. 5A-C: Yields for: (A) Full mature virus particles (VP4) in elution pool E3, and strip pool and their mass balance for resin Poros™ 50 HS as a function of the pH; (B) Full mature virus particles (VP4) and empty procapsids (VP0) in elution pool E3 for all resins and conditions tested; (C) Full mature virus particles (VP4) in elution pool E3, and strip pool and their mass balance for alternative cation exchange (CEX) and anion exchange (AEX) resins. Error bars in (A)-(C) denote ±1 standard deviation. Results shown in (A)-(C) used material generated through upstream process B.

FIG. 6A-B: Retention trends of: (A) Main elution peak in salt gradient as function of salt level in gradient; and (B) Elution salt as function of pH. In (A) each line corresponds to an average of duplicates and they depict the elution trends across all tested pHs for cation exchange (CEX) resin Poros™ HS. Anion exchange (AEX) resin Nuvia HP-Q was included for comparison purposes. In (B) each elution salt was determined from (A) by identifying the salt level at the maximum of each elution peak for the resin Poros™ 50 HS. Results shown in (A), (B) used material generated through upstream process B.

FIG. 7A-G: SDS-PAGE gels showing the Load (affinity chromatography elution product diluted 3-fold in concentrated binding buffer) and fractions for cation exchange (CEX) resin Poros™ 50 HS at: (A) pH 3.8 and 0.45 M NaCl; (B) pH 4.0 and 0.45 M NaCl; (C) pH 4.2 and 0.3 M NaCl; (D) pH 4.5 and 0.05 M NaCl; (E) pH 5.0 and 0.05 M NaCl; (F) pH 6.0 and 0.05 M NaCl. The contents of each lane per gel are shown in (G). Band VP0 is characteristic of empty procapsids alone whereas band VP2 is characteristic of full mature virus particles alone. Results shown in (A)-(G) used material generated through upstream process B.

FIG. 8A-H: Poros HS 50 chromatography traces obtained by plotting the measured absorbance of the collected fractions at 260 nm, and normalizing it over the difference between the optical absorbances at 990 nm and 900 nm, against the number of the collected fractions, or CVs, for conditions: (A) pH 3.8 and 1 M NaCl; (B) pH 4.0 and 1 M NaCl; (C) pH 4.5 and 0.7 M NaCl; (D) pH 5.0 and 0.425 M NaCl; (E) pH 3.8 and 1 M NaCl and a strip at 1.5 M NaCl; (F) pH 4.5 and 0.55 M NaCl; (G) pH 4.5 and 0.6 M NaCl; (H) pH 4.5 and 0.65 M NaCl. In (A)-(D) the solid lines with open square (□) and open circle (∘) markers denote duplicated measurements. In (E)-(H) the solid line with open circle (∘) marker denotes single measurement. Results shown in (A)-(H) used material generated through upstream process B.

FIG. 9A-G: SDS-PAGE gels showing the Load (affinity chromatography elution product adjusted to match binding conditions) and fractions for cation exchange (CEX) resin Poros HS 50 at: (A) pH 3.8 and 1 M NaCl; (B) pH 4.0 and 1 M NaCl; (C) pH 4.5 and 0.7 M NaCl; (D) pH 5.0 and 0.425 M NaCl; (E) pH 3.8 and 1 M NaCl, strip at 1.5 M NaCl (Lanes 2-5) and pH 4.5 and 0.55 M NaCl (Lanes 6-9); (F) pH 4.5 and 0.6 M NaCl (Lanes 2-5) and pH 4.5 and 0.65 M NaCl (Lanes 6-9). The contents of each lane per gel are shown in the table. Band VP0 is characteristic of empty procapsids alone whereas band VP2 is characteristic of full mature virus particles alone. Results shown in (A)-(F) used material generated through upstream process B.

FIG. 10: Yields for full mature virus particles (VP4) and empty procapsids (VP0) in flow through pool when running the resin Poros HS 50 in flowthrough mode. Error bars denote ±1 standard deviation. Results shown used material generated through upstream process B.

FIG. 11A-D: SDS-PAGE gels showing the Load (affinity chromatography elution product adjusted to match binding conditions) and flow through fraction pools for cation exchange (CEX) resin Poros HS 50 run in flowthrough mode at: (A) pH 4.5 and 0.55 M NaCl; (B) pH 4.5 and 0.6 M NaCl; (C) pH 4.5 and 0.65 M NaCl; (D) pH 4.5 and 0.7 M NaCl. In each of (A)-(D) lane 1 is the ladder and lane two is the Load. Lanes 3-15 are pooled fractions collected during the loading (flowthrough) with the size of the pool increasing every two CVs per lane (e.g., lane 3 corresponds to a pool of flow through fractions collected from 0-2 column volumes, lane 4 to a pool of fractions collected from 0-4 column volumes, and lane 15 to a pool of fractions collected from 0-20 column volumes). Results shown in (A)-(D) used material generated through upstream process B.

FIG. 12A-D: Results from column challenge study performed by spiking affinity chromatography (AC) product with large amounts of BSA and λ DNA. (A) SDS-PAGE analysis of AC product before and after the addition of the BSA and λ DNA spikes across a range of conditions. (B) The left Y-axis shows overlaid traces plotted against the number of collected fractions as generated by the Bradford (Protein) and PicoGreen (dsDNA) assays. (C) As in (A) with focus on the elution of the column; (D) The left Y-axis shows % Yields calculated relative to the loaded spiked amounts of BSA (1.2 mg) and λ DNA (2.4 μg), using the Bradford (Protein) and PicoGreen (dsDNA) assays, and relative to the loaded amount of full mature virus particles using the anti-VP4 quantitative western assay. The right Y-axis shows the amount of eluted BSA based on the BSA quantitative western assay. In (B) and (C) the lines with open square (□) and filled circle (●) markers denote the PicoGreen (dsDNA) and Bradford (Protein) assay measurements per fraction whereas the dashed line (-) denotes the salt concentration per fraction (right Y-axis). In (D) the line with filled triangle (▴) marker denotes the BSA quantitative western assay results. In (B)-(D) FT, W, E1, E2, E3, E and S denote pools of fractions spanning the fractions indicated by the double headed arrows (↔) in (B) and (C). In (D) the pool estimates from the PicoGreen (dsDNA) and Bradford (Protein) assays are based on the results of the individual fraction analysis. Results shown in (A)-(D) used material generated through upstream process A.

FIG. 13A-F: SDS-PAGE gels of affinity chromatography elution product (Feed), its 3-fold dilution in concentrated binding buffer (Load), and of elution pool (E3) and strip pool (S) for cation exchange (CEX) resins: (A) Capto S ImpAct; (B) Capto SP ImpRes; (C) Nuvia HR-S; (D) Nuvia S; (E) Capto S. In each of gels (A)-(E) lanes 6-9 are duplicates of lanes 2-5. The contents of each lane per gel are shown in (F). Band VP0 is characteristic of empty procapsids alone whereas band VP2 is characteristic of full mature virus particles alone. Results shown in (A)-(F) used material generated through upstream process B.

FIG. 14: Retention trends of five alternative cation exchange (CEX) resins run at a pH of 4.0 and 50 mM NaCl. Each elution salt was determined by identifying the salt level at the maximum of each elution peak for each CEX resin. Results shown used material generated through upstream process B.

FIG. 15A-C: SDS-PAGE gels for anion exchange (AEX) resin Nuvia HP-Q for: (A) Affinity chromatography (AC) elution product (Feed), its 3-fold dilution in concentrated binding buffer (Load), and of elution pool (E3) and strip pool (S); and (B) Affinity chromatography elution product (Feed), the 3-fold dilution of the AC elution product in concentrated binding buffer (Load), and of fractions 35-41. In (A) lanes 6-9 are duplicates of lanes 2-5. The contents of each lane per gel are shown in (C). Band VP0 is characteristic of empty procapsids alone whereas band VP2 is characteristic of full mature virus particles alone. Results shown in (A)-(C) used material generated through upstream process B.

FIG. 16A-B: SDS-PAGE gels of GSH affinity chromatography purification of multiple enterovirus serotypes in Table 4. (A) Clarified cell culture harvests and (B) GSH elution product. Lanes 1-7 correspond to Echovirus 1, Rhinovirus 1B, Rhinovirus 35, Coxsackievirus A 13, Coxsackievirus A 15, Coxsackievirus A 18, Coxsackievirus A 20b. Lanes 8 and 9 show the elution pool of for purified Coxsackievirus A 21 produced from upstream process A and D respectively.

FIG. 17: SDS-PAGE gels of GSH affinity chromatography of CVA21 produced with different upstream conditions. Clarified bulk (CB) prediluted 100× and GSH Elution (GSH) loaded neat samples shown for Experiment Arms 1-5. Viral protein (VP) bands in GSH elution samples identified as VP0, VP1, VP2, and VP3. Higher VP0 detected in Arms A, C, and D indicate differences in empty procapsid clearance across the GSH chromatography step.

FIG. 18: Comparison of the capillary electrophoresis quantitative western VP0/VP4 signal ratio detected with an anti-VP4 pAb for clarified harvest and GSH elution samples from Arms 1-5 relative to ultracentrifugation purified virus. Differences in empty procapsid/full mature virus particle ratio as estimated by VP0/VP4 ratio observed in the GSH elution samples indicate differences in empty procapsid clearance across the GSH chromatography step.

FIG. 19: A scalable and robust enterovirus purification process involving a clarification of cell culture harvest, an optional lysis step prior to harvest, the GSH affinity chromatography step, an optional anion exchange (AEX) polishing chromatography step, a solution adjustment, the cation exchange (CEX) chromatography step, a buffer exchange step using either tangential flow filtration (TFF) or size exclusion chromatography (SEC), and a final filtration step is described. The sample name for the product from each unit operation that is forwarded to the next step is shown.

FIG. 20: SDS-PAGE gels of purification process in FIG. 19 using Batch 4 as an example with GSH, AEX, solution adjustment, CEX, TFF, and filtration steps to produce purified virus. All samples loaded neat. VP0 detected in GSH elution, AEX FT, and CEX Feed samples, but is cleared in the CEX elution. The CEX strip contains mostly empty procapsids with high VP0 content. Final purified virus has high purity with only VP1, VP2, VP3 bands detected.

FIG. 21A-B: SDS-PAGE analysis of fractions collected during sucrose gradient analysis for (A) Batch 4 starting material which was purified by the cation exchange (CEX) polishing step; and (B) Batch 4 elution product pool from the CEX step. In both (A) and (B) the second lane shows the sample that was analyzed by sucrose gradient and lanes B1-B12 show the fractions collected during their sucrose gradient analysis. In both (A) and (B) the used material was generated through upstream process B.

FIG. 22A-B: Cation exchange step using resin Poros™ 50 HS at large scale. (A) Chromatographic trace from Batch 4 at 280 nm on the left-hand side Y-axis and conductivity trace on the right-hand side Y-axis. The X-axis represents column volumes (CVs); (B) SDS-PAGE analysis showing the purity of chromatography products across a 3-column purification train. In (B) samples were concentrated 10-fold before they were analyzed. Results shown in (A) used material generated through upstream process B whereas results shown in (B) used material generated through upstream process A.

FIG. 23A-E: SDS-PAGE gels for cation exchange (CEX) purification of enteroviruses using resin Capto S ImpAct for: (A) Coxsackievirus A13 (CVA13); (B) Coxsackievirus A15 (CVA15); (C) Coxsackievirus A18 (CVA18); (D) Human Rhinovirus 1B (RV1B); and (E) Human Rhinovirus 35 (RV35). In each of (A)-(E) lanes 1 and 15 is the ladder, lane 2 is the Load (affinity chromatography elution product adjusted to match binding conditions of pH 4.0 and 0.1 M NaCl). Lanes 3-14 correspond to fractions collected at the Loading (flow through), Wash, Elution 1-8 and Strip steps respectively. The observed “speckling” between 50-200 kDa resulted from the over-development of the gels due to the low protein concentration of the analyzed samples. In each of (A)-(E) the three most prominent bands were attributed to viral proteins (VP) 1, 2 and 3 (i.e., VP1, VP2 and VP3).

### DETAILED DESCRIPTION OF THE INVENTION

The invention described here relates to a scalable cation exchange chromatography process for the purification of enteroviruses (i.e., Coxsackievirus A21, CVA21), including full mature virus particles, empty procapsids, and host cell proteins from a downstream process intermediate. The cation exchange chromatography (CEX) step can be run in bind and elute, or flow-through mode. The CEX purification process can be preceded by a glutathione-based affinity chromatography step followed by an anion exchange flowthrough step.

### Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

The term “about”, when modifying the quantity (e.g., mM, or M), potency (genome/pfu, particle/pfu), purity (ng/ml), ratio of a substance or composition, the pH of a solution, or the value of a parameter characterizing a step in a method, or the like refers to variation in the numerical quantity that can occur, for example, through typical measuring, handling and sampling procedures involved in the preparation, characterization and/or use of the substance or composition; through instrumental error in these procedures; through differences in the manufacture, source, or purity of the ingredients employed to make or use the compositions or carry out the procedures; and the like. In certain embodiments, “about” can mean a variation of ±0.1%, 0.5%, 1%, 2%, 3%, 4%, 5%, or 10%. In one embodiment, “about” can mean a variation of ±10%.

As used herein, “x % (w/v)” is equivalent to x g/100 ml (for example, 5% w/v equals 50 mg/ml).

“CVA21” refers to Coxsackievirus A 21. One skilled in the art would understand that viruses may undergo mutation when cultured, passaged or propagated. The CVA21 may contain these mutations. Examples of CVA21 include but are not limited to the Kuykendall strain (GenBank accessions nos. AF546702 and AF465515), and Coe strain [9] with or without mutations. The CVA21 may be a homogenous or heterogeneous population with none, or one or more of these mutations.

When referring to the genus or species of enteroviruses, one skilled in the art would understand that viruses may undergo mutation when cultured, passaged or propagated. The enterovirus may contain these mutations. Examples of the specific enteroviruses include but are not limited to the those listed in GenBank or UnitPro data bases with or without mutations. The enterovirus may be a homogenous or heterogeneous population with none, or one or more of these mutations.

“Stationary phase” is meant any surface to which one or more ligands can immobilize to. The stationary phase may be a suspension, purification column, a discontinuous phase of discrete particles, plate, sensor, chip, capsule, cartridge, resin, beads, monolith, gel, a membrane, or filter etc. Examples of materials for forming the stationary phase include mechanically stable matrices such as porous or non-porous beads, inorganic materials (e.g., porous silica, controlled pore glass (CPG) and hydroxyapatite), synthetic organic polymers (e.g., polyacrylamide, polymethylmethacrylate, polystyrene-divinylbenzene, poly(styrenedivinyl)benzene, polyacrylamide, ceramic particles and derivatives of any of the above) and polysaccharides (e.g., cellulose, agarose and dextran). See for examples.

By “binding” an enterovirus to a stationary phase is meant exposing the enterovirus of interest to the stationary phase under appropriate conditions (pH and/or conductivity) such that the enterovirus is reversibly associated with the stationary phase by interactions between the enterovirus and ligand immobilized on the stationary phase.

The term “equilibration solution” refers to a solution to equilibrate the stationary phase prior to loading the enterovirus on the stationary phase. The equilibration solution can comprise one or more of a salt and buffer, and optionally a surfactant. In one embodiment, the equilibration solution is the same condition as the loading solution comprising the enterovirus.

The term “loading solution” is the solution which is used to load the composition comprising the enterovirus of interest and one or more impurities onto the stationary phase. The loading solution may optionally further comprise one or more of a buffer, salt and surfactant.

The term “wash solution” when used herein refers to a solution used to wash or re-equilibrate the stationary phase, prior to eluting the enterovirus of interest. For washing, the conductivity and/or pH of the wash solution is/are such that the impurities (such as empty enterovirus pro-capsid, BSA, or HCP etc.) are removed from the stationary phase. For re-equilibration, the wash solution and elution solution may be the same, but this is not required. The wash solution can comprise one or more of a salt and buffer, and optionally a surfactant such as PS-80.

The “elution solution” is the solution used to elute the enterovirus of interest from the stationary phase. The elution solution can comprise one or more of a salt, or buffer, optionally a surfactant. The presence of one or more of free reduced glutathione (GSH), salt, buffer of the elution solution is/are such that the enterovirus of interest is eluted from the stationary phase.

A “strip solution” is a solution used to dissociate strongly bound components from the stationary phase prior to regenerating a column for re-use. The strip solution has a conductivity and/or pH as required to remove substantially all impurities and the enterovirus from the stationary phase. The strip solution can comprise one or more of a salt, buffer and GSH, and optionally a surfactant and/or reducing agent.

The term “conductivity” refers to the ability of an aqueous solution to conduct an electric current between two electrodes. In solution, the current flows by ion transport. Therefore, with an increasing amount of ions present in the aqueous solution, the solution will have a higher conductivity. The unit of measurement for conductivity is mS/cm, and can be measured using a conductivity meter sold, e.g., within the GE Healthcare Äkta™ System. The conductivity of a solution may be altered by changing the concentration of ions therein. For example, the concentration of a buffering agent and/or concentration of a salt (e.g. NaCl or KCl) in the solution may be altered in order to achieve the desired conductivity. Preferably, the salt concentration of the various buffers is modified to achieve the desired conductivity as in the Examples below.

By “purifying” an enterovirus of interest or “purified composition” is meant increasing the degree of purity of the enterovirus in the composition by removing (completely or partially) at least one impurity from the composition. The impurity can be empty procapsids, BSA, host cell components such as serum, proteins or nucleic acids, cellular debris, growth medium etc. The term is not intended to refer to a complete absence of such biological molecules or to an absence of water, buffers, or salts or to components of a pharmaceutical formulation that includes the enterovirus.

As used herein, “glutathione is immobilized to a stationary phase” refers to a glutathione covalently attached to a stationary phase through conjugation of one or more reactive groups. In one embodiment, the glutathione stationary phase is a glutathione conjugated to the stationary phase through the thiol group of the glutathione.

“Surfactant” is a surface active agent that is amphipathic in nature.

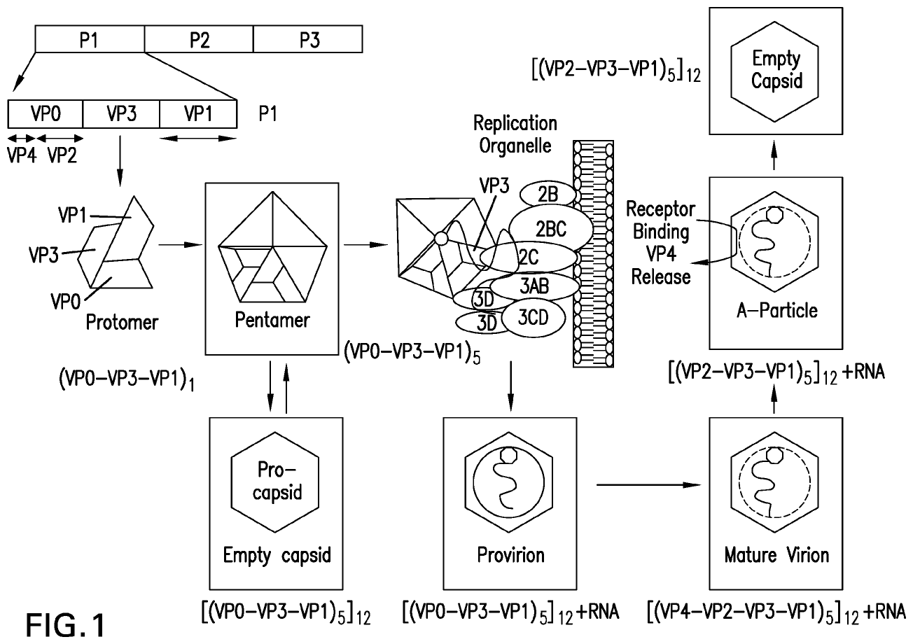

“Mature virion” “full mature virion”, “full mature virus” or “full mature virus particle”, “full mature enterovirus”, “mature enterovirus”, “mature virus particle” refers to the mature enterovirus virion [(VP4-VP2-VP3-VP1)₅]₁₂+RNA as described in FIG. 1. Examples of the CVA21 VP1-VP4 sequence is in UnitPro Data Base accession no. P22055.

“Empty capsid” refers to procapsid [(VP0-VP3-VP1)₅]₁₂, or degraded A-particle [(VP2-VP3-VP1)₅]₁₂ according to FIG. 1. An example of the VP0 sequence of CVA21 is in UnitPro Data Base accession no. P22055.

“Full capsid” refers to mature virion or provirion [(VP0-VP3-VP1)₅]₁₂+RNA as described in FIG. 1.

“Impurity” refers to a material different from the desired enterovirus. The impurity can be a serum (i.e. BSA), Host Cell Protein (HCP), Host Cell DNA (HC-DNA), non-infectious virus-related particles including VP0-containing enterovirus (protomers, pentamers, provirions, procapsids), VP2-containing enterovirus (A-particles, or degraded A-particles). In one embodiment, the desired enterovirus is full mature enterovirus (e.g. full mature CVA21).

### Cation Exchange Chromatography

The invention provides a method of purifying an enterovirus comprising the steps of:
a. binding the enterovirus to a cation exchange stationary phase using a loading solution with a pH of about 3.5 to 6.0;
b. eluting the enterovirus from the stationary phase with an elution solution with a pH of about 3.5 to 4.8.

In one embodiment, prior to step (a), equilibrating the stationary phase with an equilibration solution is performed.

In another aspect of the method, after step (a) but prior to step (b), it further comprises step (i) of washing the stationary phase with one or more wash solutions. In one embodiment, one or more impurities are removed from the wash step. In another embodiment, step (i) comprises a wash step with a wash solution having a conductivity higher than the equilibration solution or loading solution. In a further embodiment, the conductivity of the loading or equilibration solution is the same as the wash solution in the wash step.

Various commercially available cation ion exchange stationary phases may be used in the invention. Examples include but are not limited to Poros™ 50 HS (ThermoFisher Scientific, MA, USA), Capto™ S ImpAct (Cytiva Life Sciences, Uppsala, Sweden), Capto™ SP ImpRes (Cytiva Life Sciences), or Nuvia™ HR-S (Bio-Rad, CA, USA). In one embodiment, the stationary phase is Poros™ 50 HS. In another embodiment, the cation ion exchange ligand is a sulfonic acid (SO₃⁻) functional group. The functional group can be C₁-C₆alkylSO₃⁻ (Poros 50 HS, Capto S, Capto S ImpAct, Capto SP ImpRes) or a sulfonic acid (SO₃⁻) attached to a polymeric surface extender (Nuvia S and Nuvia HR-S). In one embodiment, the resin bead diameter is 30-70 μm. In another embodiment, the resin bead diameter is 30-60 μm. In another embodiment, the resin bead diameter is 40-50 μm.

In one embodiment, the loading solution, equilibration solution, wash solution or elution solution comprises a salt, preferably a monovalent metal ion salt, such as NaCl or KCl. In another embodiment, the loading solution or equilibration solution comprises about 50-500 mM NaCl or KCl. In another embodiment, the loading solution or equilibration solution comprises up to about 350 mM or 400 mM NaCl or KCl. In another embodiment, the loading solution or equilibration solution comprises about 400 mM NaCl or KCl.

In one embodiment, the wash solution comprises about 50-600 mM NaCl or KCl. In one embodiment, the wash solution comprises about 100-600 mM NaCl or KCl. In another embodiment, the wash solution comprises about 350-450 mM NaCl or KCl. In another embodiment, the wash solution comprises about 400-500 mM NaCl or KCl. In a further embodiment, the wash solution comprises about 500 mM NaCl or KCl.

The elution step may be performed with a solution with high ionic strength or high conductivity, and low pH (for example pH about 3.5-4.8). In one embodiment, the elution solution comprises about 350-1200 mM of monovalent salt. In one embodiment, the elution solution comprises about 300-900 mM of monovalent salt. In one embodiment, the elution solution comprises about 200-1000 mM of monovalent salt. In one embodiment, the elution solution comprises about 550-850 mM of NaCl or KCl. In another embodiment, the elution solution comprises about 800 mM NaCl, and optionally about 0.001-1% w/v PS-80. In yet a further embodiment, the elution solution comprises about 800 mM NaCl, and about 0.005% w/v PS-80.

In one embodiment, one or more of the loading solution, equilibration solution, wash solutions and elution solution has a pH of about 3.5-4.8. In another embodiment, one or more of the loading solution, equilibration solution, wash solutions and elution solution has a pH of about 3.8-4.5. In another embodiment, one or more of the loading solution, equilibration solution, wash solutions and elution solution has a pH of about 3.5-4.5. In another embodiment, one or more of the loading solution, equilibration solution, wash solutions and elution solution has a pH of about 4.2-4.8. In a another embodiment, one or more of the loading solution, equilibration solution, wash solutions and elution solution has a pH of about 4. In a further embodiment, one or more of the loading solution, equilibration solution and wash solutions has a pH of about 3.5-6.0, and the elution solution has a pH of about 3.5-4.8. In a further embodiment, one or more of the loading solution, equilibration solution and wash solutions has a pH of about 3.5-6.0, and the elution solution has a pH of about 3.8-4.5. In a further embodiment, one or more of the loading solution, equilibration solution and wash solutions has a pH of about 3.5-6.0, and about 50-500 mM monovalent salt, and the elution solution has a pH of about 3.8-4.5, and about 350-1200 mM monovalent salt. In a further embodiment, one or more of the loading solution, equilibration solution and wash solutions has a pH of about 3.5-6.0, and about 50-500 mM monovalent salt, and the elution solution has a pH of about 3.8-4.5, and about 200-1000 mM monovalent salt.

In one embodiment, one or more of the loading solution, equilibration solution, wash solutions and elution solution further comprises a surfactant. In another embodiment, the surfactant is PS-80 or PS-20. In another embodiment, the surfactant is about 0.001-1% w/v PS-80. In another embodiment, the surfactant is about 0.001-0.1% w/v PS-80. In another embodiment, the surfactant is about 0.005% w/v PS-80.

In a another embodiment, the loading and equilibration solution has a pH of about 3.8-4.5, comprises about 350-450 mM NaCl or KCl, optionally about 0.001-0.1% w/v PS-80; the wash solution has a pH of about 3.8-4.5, comprises about 450-550 mM NaCl or KCl, optionally about 0.001-0.1% w/v PS-80; and the elution solution has a pH of about 3.8-4.5, comprises about 700-900 mM NaCl or KCl, and optionally about 0.001-0.1% w/v PS-80. In a preferred embodiment, the loading and equilibration solution comprises 50 mM citrate, pH 4.0, 400 mM NaCl, 0.005% w/v PS-80; the wash solution comprises 25 mM citrate, pH 4.0, 500 mM NaCl, w/v PS-80; and the elution solution comprises 25 mM citrate, pH 4.0, 800 mM NaCl, and 0.005% w/v PS-80.

The invention provides a method of purifying an enterovirus comprising the steps of:
a. applying a loading solution comprising the enterovirus to a cation exchange stationary phase using a loading solution with a pH of about 3.5 to 4.7;
b. collecting the flow-through comprising the enterovirus.

In one embodiment, prior to step (a), the stationary phase is equilibrated with an equilibration solution.

In another aspect of the method, it further comprises step (i) of washing the stationary phase with one or more wash solutions after step (b) and further collecting the flow-through of the wash solutions. In one embodiment, one or more of the loading solution, equilibration solution, and wash solution has a pH of about 3.5-4.5. In another embodiment, one or more of the loading solution, equilibration solution, and wash solution has a pH of about 3.8-4.0. In a further embodiment, one or more of the loading solution, equilibration solution, and wash solution has a pH of about 3.8.

In another aspect of the method, one or more of the loading solution, equilibration solution, and wash solution comprises about 400-1500 mM monovalent salt. In one embodiment, one or more of the loading solution, equilibration solution, and wash solution comprises about 350-800 mM monovalent salt (e.g. NaCl or KCl). In one embodiment, one or more of the loading solution, equilibration solution, and wash solution comprises about 900-1100 mM monovalent salt (e.g. NaCl or KCl), and has a pH of about 3.5-4.0 or 3.8-4.0. In one embodiment, one or more of the loading solution, equilibration solution, and wash solution comprises about 550-700 mM monovalent salt (e.g. NaCl or KCl), and has a pH of about 4.0-4.7 or 4.0-4.5. In one embodiment, one or more of the loading solution, equilibration solution, and wash solution comprises about 450-800 mM monovalent salt (e.g. NaCl or KCl), and has a pH of about 4.0-4.7 or 4.0-4.5. In one embodiment, the loading solution has the same conductivity as the equilibration solution or wash solution.

In another aspect of the method, one or more of the loading solution, equilibration solution, and wash solution further comprises a surfactant. In another embodiment, the surfactant is PS-80 or PS-20. In another embodiment, the surfactant is about 0.001-1% w/v PS-80. In another embodiment, the surfactant is about 0.001-0.1% w/v PS-80. In another embodiment, the surfactant is about 0.005% w/v PS-80.

In one embodiment, the desired enterovirus is full mature enterovirus. In one embodiment, the desired enterovirus is Coxsackievirus. In one embodiment, the desired enterovirus is full mature CVA21. In one embodiment, at least the full mature enterovirus binds to the stationary phase upon loading the solution. In one embodiment, the purification process removes one or more impurities such as serum (i.e. BSA), HCP, HC-DNA, non-infectious virus-related particles including but not limited to VP0-containing enterovirus (protomers, pentamers, provirions, procapsids), VP2-containing enterovirus (A-particles, or empty capsids from degraded A-particles). In a further embodiment, the purification process removes enterovirus empty procapsids (e.g., CVA21 empty procapsids).

### Glutathione Affinity Chromatography

The CEX purification method of the invention can be preceded by glutathione affinity (GSH) chromatography. After conducting GSH chromatography in bind and elute mode, the GSH elution product after solution adjustment, can be loaded to the CEX stationary phase. Alternatively, the GSH elution product (with or without solution adjustment) can be loaded to an anion exchange stationary phase, the flow-through collected; and after solution adjustment, applied to the CEX stationary phase. In one embodiment, the glutathione affinity chromatography stationary phase comprises a glutathione (GSH) immobilized to the surface of a stationary phase. Glutathione (also named L-glutathione, reduced glutathione, or GSH) is a biologically-active tri-peptide (glutamic acid-cysteine-glycine) in human cells used to control redox potential and is involved in many cellular functions [11]. GSH has the following chemical structure and name:

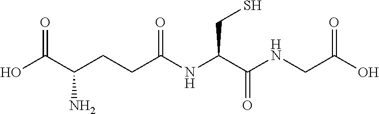 [SMILES: N[C@@H](CCC(=O)N[C@@H](CS)C(=O)NCC(=O)O)C(=O)O]

### (2S)-2-amino-5-[[(2R)-1-(carboxymethylamino)-1-oxo-3-sulfanylpropan-2-yl]amino]-5-oxopentanoic acid

The glutathione can be immobilized to the stationary phase through conjugation of the SH group using maleimide, haloacetyl, pyridyl disulfide, epoxy or other similar sulthydryl-reactive based chemistries. See for examples. GSH resin is also commercially available through several vendors (Cytiva Life Sciences, ThermoFisher Scientific, Qiagen, Sigma).

In batch mode, the stationary phase is utilized free in solution. For utilization in flow mode, the stationary phase is packaged into a column, capsule, cartridge, filter or other support and a flowrate of about 1-500 cm/hr is used.

In one aspect, the invention provides a method of purifying an enterovirus comprising the steps of:
a. binding an enterovirus to a stationary phase using a loading solution, wherein glutathione is immobilized to the stationary phase;
b. eluting the enterovirus from the stationary phase with an elution solution;
c. binding the eluted enterovirus to a cation exchange stationary phase using a loading solution with a pH of about 3.5 to 6.0;
d. eluting the enterovirus from the stationary phase with an elution solution with a pH of about 3.5 to 4.8.

In one embodiment, prior to step (a), equilibrating the stationary phase with an equilibration solution is performed. In one embodiment, one or more impurities are in the flowthrough of step (a).

In another aspect of the method, after step a) but prior to step (b), it further comprises step i) of washing the stationary phase with one or more wash solutions. In one embodiment, one or more impurities are removed from the wash step. In another embodiment, step (i) comprises a first wash step with a wash solution having a conductivity higher than the equilibration solution or loading solution. In another embodiment, step (i) comprises a second wash step with a wash solution having a conductivity lower than the wash solution in the first wash step. In a further embodiment, the conductivity of the elution solution is the same as the wash solution in the second wash step.

In one embodiment of the GSH chromatography steps a) and b), the loading solution, equilibration solution, wash solution or elution solution comprises a salt, preferably a monovalent metal ion salt, such as NaCl or KCl. In another embodiment, the loading solution or equilibration solution comprises about 50-200 mM NaCl or KCl. In a another embodiment, the loading solution or equilibration solution comprises about 100 mM NaCl or KCl.

In one embodiment of the GSH chromatography steps a) and b), the wash solution comprises about 50-400 mM NaCl or KCl. In another embodiment, the wash solution comprises about 350-450 mM NaCl or KCl. In another embodiment, the wash solution comprises about 400-500 mM NaCl or KCl. In a further embodiment, the wash solution comprises about 400 mM NaCl or KCl. In a further embodiment, a first wash solution comprises about 100-500 mM NaCl or KCl and a second wash solution comprises about 50-500 mM NaCl or KCl. In a further embodiment, a first wash solution comprises about 350-500 mM NaCl or KCl and the second wash solution comprises about 50-150 mM NaCl or KCl. In a further embodiment, the first wash solution comprises about 400 mM NaCl or KCl and the second wash solution comprises about 75 mM NaCl or KCl. In a further embodiment, the second wash solution comprises about 50-150 mM NaCl or KCl. In a further embodiment, the second wash solution comprises about 100 mM NaCl or KCl.

The elution of the GSH chromatography step may be performed with a solution with high ionic strength or high conductivity, low pH (for example pH about 5-7), or in the presence of free GSH, or a combination thereof. In one embodiment, the elution solution comprises about 0.5-1 M of monovalent salt such as NaCl or KCl. In one embodiment, the elution solution comprises about 0.5 M of NaCl or KCl. In one embodiment, the elution solution comprises about 50-500 mM of NaCl or KCl. In another embodiment, the elution solution comprises about 0.1-100 mM glutathione. In another embodiment, the elution solution comprises about 0.1-50 mM glutathione. In another embodiment, the elution solution comprises about 0.1-25 mM glutathione. In another embodiment, the glutathione in the elution solution is about 1 mM. In one embodiment, the elution solution comprises about 0.5-5 mM glutathione and about 75-150 mM NaCl or KCl. In one embodiment, the elution solution comprises about 0.5-25 mM glutathione and about 50-500 mM NaCl or KCl. In another embodiment, the elution solution comprises about 0.1-100 mM glutathione and about 75-150 mM NaCl, and optionally about 0.001-1% w/v PS-80. In yet a further embodiment, the elution solution comprises about 100 mM NaCl, about 1 mM glutathione, and about 0.005% w/v PS-80.

In one embodiment of the GSH chromatography steps a) and b), one or more of the loading solution, equilibration solution, wash solutions and elution solution has a pH of about 6.5-8.5. In a another embodiment, one or more of the loading solution, equilibration solution, wash solutions and elution solution has a pH of about 7-8. In a another embodiment, one or more of the loading solution, equilibration solution, wash solutions and elution solution has a pH of about 8. In a further embodiment, one or more of the loading solution, equilibration solution, wash solutions and elution solution has a pH of about 6-9. In yet a further embodiment, one or more of the loading solution, equilibration solution, wash solutions and elution solution has a pH of about 5-10.

In one embodiment of the GSH chromatography steps a) and b), one or more of the loading solution, equilibration solution, wash solutions and elution solution further comprises a surfactant. In another embodiment, the surfactant is PS-80 or PS-20. In another embodiment, the surfactant is about 0.001-1% w/v PS-80. In another embodiment, the surfactant is about w/v PS-80. In another embodiment, the surfactant is about 0.005% w/v PS-80. In one embodiment, one or more of the loading solution, wash solutions and elution solution further comprises EDTA, or a reducing agent such as DTT or ß-mercaptoethanol. In another embodiment, the reducing agent is DTT. In another embodiment, the DTT is at about 0.1-10 mM. In another embodiment, the DTT is at about 0.1-5 mM. In another embodiment, the DTT is at about 1 mM.

Embodiments of the CEX chromatography steps in c) and d) were described in the CEX section above. The methods of the invention can be used in conjunction with other chromatography or purification steps to remove impurities. In one embodiment, after step b) but prior to step c) above, comprises the steps of
(1) loading the eluted enterovirus to an anionic exchange stationary phase using a loading solution,
(2) collecting the enterovirus from the flow-through.

In one embodiment, the loading solution in step 1) comprises about 50-500 mM monovalent salt concentration at pH about 6-9.

In another aspect, the invention provides a method of purifying Coxsackievirus (e.g. CVA21) comprising the steps of:
a. binding the Coxsackievirus to a stationary phase using a loading solution that has a pH of about 6-9, wherein glutathione is immobilized to the stationary phase;
b. washing the stationary phase with a wash solution comprising about 100-500 mM NaCl or KCl, optionally about 0.5-5 mM DTT, optionally about 0.001-0.1% w/v PS-80, and pH about 7-9,
c. optionally, washing the stationary phase with a wash solution comprising about 50-500 mM NaCl or KCl, optionally about 0.5-5 mM DTT, optionally about 0.001-0.1% w/v PS-80, and pH about 7-9,
d. eluting the Coxsackievirus from the stationary phase with an elution solution comprising about 50-600 mM NaCl or KCl, about 0.1-25 mM glutathione, optionally about 0.5-5 mM DTT, optionally about 0.001-0.1% w/v PS-80, and pH about 7-9,
e. binding the eluted Coxsackievirus to a cation exchange stationary phase using a loading solution with a pH of about 3.8 to 4.5, comprising about 50-500 mM NaCl or KCl, optionally about 0.001-0.1% w/v PS-80;
f. washing the stationary phase with a wash solution comprising about 50-500 mM NaCl or KCl, optionally about 0.001-0.1% w/v PS-80, and pH about 3.8 to 4.5;
g. eluting the Coxsackievirus from the stationary phase with an elution solution comprising about 200-1000 mM NaCl or KCl, optionally about 0.001-0.1% w/v PS-80, with a pH of about 3.8 to 4.5.

In one embodiment, after step d) but prior to step e) above, comprises the steps of
(1) loading the eluted enterovirus to an anionic exchange stationary phase using a loading solution,
(2) collecting the enterovirus from the flow-through.
   In one embodiment, the loading solution in step 1) comprises about 50-500 mM monovalent salt concentration at pH about 6-9.

In another aspect, the invention provides a purified composition of the enterovirus obtainable by or produced by the foregoing purification steps and/or embodiments of the invention.

In one embodiment, the desired enterovirus is full mature enterovirus. In one embodiment, the desired enterovirus is full mature Coxsackievirus. In one embodiment, the desired enterovirus is full mature CVA21. In one embodiment, at least the full mature enterovirus binds to the stationary phase upon loading the solution. In one embodiment, the purification process removes one or more impurities such as serum (i.e. BSA), HCP, HC-DNA, non-infectious virus-related particles including but not limited to VP0-containing enterovirus (protomers, pentamers, provirions, procapsids), VP2-containing enterovirus (A-particles, or empty capsids from degraded A-particles). In a further embodiment, the purification process removes enterovirus empty procapsids (e.g., CVA21 empty procapsids).

### Enterovirus

Any suitable source of enterovirus may be used in the methods of the invention [1]. The enterovirus particle can be poliovirus, Group A Coxsackievirus, Group B Coxsackievirus, echovirus, rhinovirus, and numbered enterovirus. In one embodiment, the enterovirus is a Group A, B or C enterovirus. In one embodiment, the enterovirus is a Group C enterovirus. In one embodiment, the enterovirus is a Group A or B Coxsackievirus. In another embodiment, the enterovirus is Group A Coxsackievirus. In one embodiment, the Group C enterovirus is a Group A Coxsackievirus selected from the group consisting of CVA1, CVA11, CVA13, CVA15, CVA17, CVA18, CVA19, CVA20a, CVA20b, CVA20c, CVA21, CVA22 and CVA24. In one embodiment, the Group A Coxsackievirus is selected from the group consisting of CVA13, CVA15, CVA18, CVA20, and CVA21. Various suitable strains of these viruses may be obtained from the American Type Culture Collection (ATCC), 10801 University Blvd., Manassas, Va. 20110-2209 USA, such as material deposited under the Budapest Treaty on the dates provided below, and is available according to the terms of the Budapest Treaty: Coxsackie group A virus, strain CVA13, ATCC No.: PTA-8854, deposited Dec. 10, 2007; Coxsackie group A virus, strain CVA15 (G9), ATCC No.: PTA-8616, deposited Aug. 15, 2007; Coxsackie group A virus, strain CVA18, ATCC No.: PTA-8853, deposited Dec. 20, 2007; and Coxsackie group A virus, strain CVA21 (Kuykendall), ATCC No.: PTA-8852, deposited Dec. 20, 2007. Other Group A Coxsackie virus under Group C enterovirus referenced in the literature include but are not limited to CVA1 (GenBank accession no. AF499635, [13]), CVA11 (GenBank accession no. AF499636), CVA17 (GenBank accession no. AF499639), CVA19 (GenBank accession no. AF499641), CVA20 (GenBank accession no. AF499642), CVA20a ([14]), CVA20b ([14]), CVA20c ([15]), CVA22 (GenBank accession no. AF499643; [14]), and CVA24 (GenBank accession no. EF026081; [16]). In a preferred embodiment, the enterovirus is a Coxsackievirus A21.

In another embodiment, the enterovirus is a Group B enterovirus. In another embodiment, the Group B enterovirus is echovirus. In another embodiment, the Group B enterovirus is echovirus-1 (EV-1). Examples of echovirus-1 include those with GenBank accession nos. AF029859, AF029859.2 and AF250874.

In another embodiment, the enterovirus is a Group B Coxsackievirus. In a further embodiment, the Group B Coxsackievirus is Coxsackievirus B3 (CVB3) or Coxsackievirus B4 (CVB4).

In a further embodiment, the enterovirus is a Rhinovirus A, B or C. In another embodiment, the enterovirus is Rhinovirus A or B. In yet a further embodiment, the enterovirus is Human Rhinovirus 14 (HRV14). In yet a further embodiment, the enterovirus is Human Rhinovirus 1B or 35. An example of Human Rhinovirus 1B is Genbank accession no. D00239.1. An example of human Rhinovirus 35 is Genbank accession no. EU870473. A summary of the current understanding of enterovirus morphogenesis is detailed in FIG. 1. Furthermore, genetically modified enterovirus with transgene insertion, and inactivated enteroviruses can be used in the methods of the invention.

### EXAMPLES

The examples are presented in order to more fully illustrate the various embodiments of the invention. These examples should in no way be construed as limiting the scope of the invention recited in the appended claims.

### Example 1: Materials and Methods

### RoboColumn Chromatography Method Description

High throughput chromatography experiments were performed using Opus® RoboColumns® (Repligen, MA, USA) on a Tecan EVO® 150 robotic station (base unit) operated by EVOware® v2.8 which was equipped with an 8-channel Liquid Handling (LiHa) arm and an eccentric Robot Manipulator (RoMa) arm (Tecan Group Ltd., Marmedorf, Switzerland). The LiHa arm was equipped with short stainless-steel tips and for the operation of the RoboColumns the robotic station was fitted with the Te-Chrom™ and Te-Shuttle™ modules (fraction collection system) and integrated with an Infinite® M1000 pro reader (Tecan Group Ltd.).

The described configuration of the robotic station allowed for up to 8 RoboColumn-based chromatographic separations to be run in parallel in a process described in [17]. A total of 12 separations were performed aiming to evaluate the separation of full mature virus particles and empty procapsids on a selection of ion exchange resins (Tables 1 and 2). The aforementioned separation was tested in a range of mobile phase conditions for cation exchange (CEX) resin Poros™ 50 HS (ThermoFisher Scientific). Additional CEX resins Capto™ S ImpAct, SP ImpRes, S (Cytiva Life Sciences) and Nuvia™ S and HR-S(Bio-Rad) were also evaluated along with the strong anion exchanger (AEX) Nuvia HP-Q (Bio-Rad). All separations (Tables 1 and 2) employed 200 μL RoboColumns and were run in bind and elute mode with a salt gradient and a residence time of 2 minutes for all phases. Each separation included an equilibration, load, wash, elution and strip phase with their durations, in terms of column volumes, shown in Tables 1 and 2. These tables also describe the composition of buffers employed in each phase of each separation. Here, the pH of these buffers across all phases, apart from the strip, remained constant and the same applied to the salt level during the equilibration, load and wash phase. Hence, each separation could be identified by the resin used and the combination of pH and salt level employed.

The CEX resin-based separations employed a Citrate buffer system with varying pH between 3.8 and 6.0 and NaCl concentration to match the desired mobile phase conditions during the equilibration, wash, and elution phases (Tables 1 and 2). Conversely, for the AEX resin, a Tris buffer system was used with a pH of 9.0 and varying NaCl concentration for the equilibration, wash and elution phases (Tables 1 and 2). In all separations, the columns were stripped using a 100 mM Tris pH 7.0, 1000 mM NaCl buffer. All separations also employed the same duration for the equilibration, wash, and strip phases (i.e., 9, 5 and 5 column volumes, CVs, respectively) whereas the CVs varied during the elution and load phases. For the former, this was accrued due to maintaining a constant salt elution gradient slope of ˜60 mM CV⁻¹. For example, for separations 1 and 2 (Table 1), the columns were eluted in 19 CVs (450 mM NaCl to 1500 mM NaCl) whereas separations 7-11 (Table 2) were run with a 24 CV gradient (50 mM NaCl to 1500 mM NaCl). Since RoboColumn experiments do not allow for an ‘on the fly’ mixing of mobile phases, the elution gradients were simulated by step gradients. Here, each step had a size of 1 CV and a salt level (Cₛₐₗₜ) determined by the equation Cₛₐₗₜ=C_{salt,o}+60×CVₑₗᵤₜᵢₒₙ, where C_{salt,o} is the starting salt level in the gradient (e.g., 50 mM NaCl or 450 mM NaCl) and CVₑₗᵤₜᵢₒₙ corresponds to the elution phase column volume number. Here, Cₛₐₗₜ, is also the salt level of the buffer used in the equilibration, load and wash phases. The steps in the gradient were generated by mixing, for each buffer system, the low (50 mM NaCl or 450 mM NaCl) and high (1000 mM NaCl) salt buffers for a given pH at different ratios to obtain the desired salt concentrations. For the loading of the columns, 60 or 30 CVs were employed (Tables 1 and 2). Here, the product pool from a preceding Affinity Chromatography (AC) step was diluted 3-fold in concentrated buffers with a composition designed to match the composition (pH, NaCl concentration and buffer system concentration) of the equilibration mobile phase buffer post dilution. For the AEX resin separation, the Tris concentration was increased to 70 mM during the load compared to 50 mM Tris at the equilibration phase.

Finally, all separations were fractionated by collecting fractions every 200 μL, or one CV. These were collected in UV transparent 96 well microplates (Corning Inc., NY, USA) and were read on a plate reader at 260 nm, 280 nm, 900 nm and 990 nm. The made measurements were employed to construct chromatographic traces and to determine how the collected fractions should be pooled and which fractions required further analysis. Here, the fractions were pooled in a fashion yielding up to five pools containing flowthrough fractions (FT1-FT5), one pool containing the wash fractions (W), and one pool containing the strip fractions (S). The fractions collected during the elution of the columns were pooled in three different ways. Pools E1 and E2 contained the fractions in approximately the first and second half of the main elution peak respectively whereas pool E3 contained all fractions included in pools E1 and E2 in addition to a few fractions flowing the complete elution of the main peak in the gradient. All pooling was carried out on the described robotic station. The analysis of these pools and individual fractions took place via analytical methods including quantitative western blotting and SDS-PAGE.

TABLE 1
Details of chromatographic conditions screening the full mature virus
particles/empty procapsids separation on RoboColumns packed with cation exchange resin
Poros ™ 50 HS.

| | | | | | Gradient | |
|---|---|---|---|---|---|---|
| Separation | Resin | Equilibration | Load | Wash | Elution | Strip |
| 1 | Poros ™ | 50 mM | 50 mM | 50 mM | 50 mM | 100 mM |
| | 50 HS | Citrate, pH | Citrate, | Citrate, pH | Citrate, pH | Tris, pH |
| | 200 μL | 3.8, 450 mM | pH 3.8, | 3.8, 450 mM | 3.8, 0.005% | 7.0, 1M |
| | | NaCl, | 450 mM | NaCl, | w/v PS-80, | NaCl, |
| | | 0.005% w/v | NaCl, | 0.005% w/v | 450 to 1500 | 0.005% |
| | | PS-80, 9 | 0.005% | PS-80, 5 | mM NaCl | w/v PS-80, |
| | | CVs | w/v PS- | CVs | in 19 CVs | 5 CVs |
| | | | 80, 60 | | | |
| | | | CVs | | | |
| 2 | Poros ™ | 50 mM | 50 mM | 50 mM | 50 mM | 100 mM |
| | 50 HS | Citrate, pH | Citrate, | Citrate, pH | Citrate, pH | Tris, pH |
| | 200 μL | 4.0, 450 mM | pH 4.0, | 4.0, 450 mM | 4.0, 0.005% | 7.0, 1M |
| | | NaCl, | 450 mM | NaCl, | w/v PS-80, | NaCl, |
| | | 0.005% w/v | NaCl, | 0.005% w/v | 450 to 1500 | 0.005% |
| | | PS-80, 9 | 0.005% | PS-80, 5 | mM NaCl | w/v PS-80, |
| | | CVs | w/v PS- | CVs | in 19 CVs | 5 CVs |
| | | | 80, 60 | | | |
| | | | CVs | | | |
| 3 | Poros ™ | 50 mM | 50 mM | 50 mM | 50 mM | 100 mM |
| | 50 HS | Citrate, pH | Citrate, | Citrate, pH | Citrate, pH | Tris, pH |
| | 200 μL | 4.2, 300 mM | pH 4.2, | 4.2, 300 mM | 4.2, 0.005% | 7.0, 1M |
| | | NaCl, | 300 mM | NaCl, | w/v PS-80 | NaCl, |
| | | 0.005% w/v | NaCl, | 0.005% w/v | 300 to 1500 | 0.005% |
| | | PS-80, 9 | 0.005% | PS-80, 5 | mM NaCl | w/v PS-80, |
| | | CVs | w/v PS- | CVs | in 19 CVs | 5 CVs |
| | | | 80, 60 | | | |
| | | | CVs | | | |
| 4 | Poros ™ | 50 mM | 50 mM | 50 mM | 50 mM | 100 mM |
| | 50 HS | Citrate, pH | Citrate, | Citrate, pH | Citrate, pH | Tris, pH |
| | 200 μL | 4.5, 50 mM | pH 4.5, | 4.5, 50 mM | 4.5, 0.005% | 7.0, 1M |
| | | NaCl, | 50 mM | NaCl, | w/v PS-80 | NaCl, |
| | | 0.005% w/v | NaCl, | 0.005% w/v | 50 to 1000 | 0.005% |
| | | PS-80, 9 | 0.005% | PS-80, 5 | mM NaCl | w/v PS-80, |
| | | CVs | w/v PS- | CVs | in 16 CVs | 5 CVs |
| | | | 80, 60 | | followed by | |
| | | | CVs | | a step at 50 | |
| | | | | | mM Citrate, | |
| | | | | | pH 4.5, | |
| | | | | | 1000 mM | |
| | | | | | NaCl, | |
| | | | | | 0.005% w/v | |
| | | | | | PS-80 for 3 | |
| | | | | | CVs | |
| 5 | Poros ™ | 50 mM | 50 mM | 50 mM | 50 mM | 100 mM |
| | 50 HS | Citrate, pH | Citrate, | Citrate, pH | Citrate, pH | Tris, pH |
| | 200 μL | 5.0, 50 mM | pH 5.0, | 5.0, 50 mM | 5.0, 0.005% | 7.0, 1M |
| | | NaCl, | 50 mM | NaCl, | w/v PS-80 | NaCl, |
| | | 0.005% w/v | NaCl, | 0.005% w/v | 50 to 1000 | 0.005% |
| | | PS-80, 9 | 0.005% | PS-80, 5 | mM NaCl | w/v PS-80, |
| | | CVs | w/v PS- | CVs | in 16 CVs | 5 CVs |
| | | | 80, 60 | | followed by | |
| | | | CVs | | a step at 50 | |
| | | | | | mM Citrate, | |
| | | | | | pH 5.0, | |
| | | | | | 1000 mM | |
| | | | | | NaCl, | |
| | | | | | 0.005% w/v | |
| | | | | | PS-80 for 3 | |
| | | | | | CVs | |
| 6 | Poros ™ | 50 mM | 50 mM | 50 mM | 50 mM | 100 mM |
| | 50 HS | Citrate, pH | Citrate, | Citrate, pH | Citrate, pH | Tris, pH |
| | 200 μL | 6.0, 50 mM | pH 6.0, | 6.0, 50 mM | 6.0, 0.005% | 7.0, 1M |
| | | NaCl, | 50 mM | NaCl, | w/v PS-80 | NaCl, |
| | | 0.005% w/v | NaCl, | 0.005% w/v | 50 to 1000 | 0.005% |
| | | PS-80, 9 | 0.005% | PS-80, 5 | mM NaCl | w/v PS-80, |
| | | CVs | w/v PS- | CVs | in 16 CVs | 5 CVs |
| | | | 80, 60 | | followed by | |
| | | | CVs | | a step at 50 | |
| | | | | | mM Citrate, | |
| | | | | | pH 6.0, | |
| | | | | | 1000 mM | |
| | | | | | NaCl, | |
| | | | | | 0.005% w/v | |
| | | | | | PS-80 for 3 | |
| | | | | | CVs | |

TABLE 2
Details of chromatographic conditions screening the full mature virus particles/empty
procapsids separation on RoboColumns packed with alternative
cation and anion exchange resins.

| | | | | | Gradient | |
|---|---|---|---|---|---|---|
| Separation | Resin | Equilibration | Load | Wash | Elution | Strip |
| 7 | Capto ™ | 50 mM | 50 mM | 50 mM Citrate, | 50 mM | 100 mM |
| | S | Citrate, pH | Citrate, | pH 4.0, 50 mM | Citrate, pH | Tris, pH |
| | ImpAct | 4.0, 50 mM | pH 4.0, 50 | NaCl, 0.005% | 6.0, | 7.0, 1M |
| | 200 μL | NaCl, | mM | w/v PS-80, 5 | 0.005% | NaCl, |
| | | 0.005% w/v | NaCl, | CVs | w/v PS-80 | 0.005% |
| | | PS-80, 9 | 0.005% | | 50 to 1500 | w/v PS- |
| | | CVs | w/v PS- | | mM NaCl | 80, 5 |
| | | | 80, 30 | | in 24 CVs | CVs |
| | | | CVs | | | |
| 8 | Capto ™ | 50 mM | 50 mM | 50 mM Citrate, | 50 mM | 100 mM |
| | SP | Citrate, pH | Citrate, | pH 4.0, 50 mM | Citrate, pH | Tris, pH |
| | ImpRes | 4.0, 50 mM | pH 4.0, 50 | NaCl, 0.005% | 6.0, | 7.0, 1M |
| | 200 μL | NaCl, | mM | w/v PS-80, 5 | 0.005% | NaCl, |
| | | 0.005% w/v | NaCl, | CVs | w/v PS-80 | 0.005% |
| | | PS-80, 9 | 0.005% | | 50 to 1500 | w/v PS- |
| | | CVs | w/v PS- | | mM NaCl | 80, 5 |
| | | | 80, 30 | | in 24 CVs | CVs |
| | | | CVs | | | |
| 9 | Nuvia ™ | 50 mM | 50 mM | 50 mM Citrate, | 50 mM | 100 mM |
| | HR-S | Citrate, pH | Citrate, | pH 4.0, 50 mM | Citrate, pH | Tris, pH |
| | 200 μL | 4.0, 50 mM | pH 4.0, 50 | NaCl, 0.005% | 6.0, | 7.0, 1M |
| | | NaCl, | mM | w/v PS-80, 5 | 0.005% | NaCl, |
| | | 0.005% w/v | NaCl, | CVs | w/v PS-80 | 0.005% |
| | | PS-80, 9 | 0.005% | | 50 to 1500 | w/v PS- |
| | | CVs | w/v PS- | | mM NaCl | 80, 5 |
| | | | 80, 30 | | in 24 CVs | CVs |
| | | | CVs | | | |
| 10 | Nuvia ™ | 50 mM | 50 mM | 50 mM Citrate, | 50 mM | 100 mM |
| | S 200 μL | Citrate, pH | Citrate, | pH 4.0, 50 mM | Citrate, pH | Tris, pH |
| | | 4.0, 50 mM | pH 4.0, 50 | NaCl, 0.005% | 6.0, | 7.0, 1M |
| | | NaCl, | mM | w/v PS-80, 5 | 0.005% | NaCl, |
| | | 0.005% w/v | NaCl, | CVs | w/v PS-80 | 0.005% |
| | | PS-80, 9 | 0.005% | | 50 to 1500 | w/v PS- |
| | | CVs | w/v PS- | | mM NaCl | 80, 5 |
| | | | 80, 30 | | in 24 CVs | CVs |
| | | | CVs | | | |
| 11 | Nuvia ™ | 50 mM | 50 mM | 50 mM Citrate, | 50 mM | 100 mM |
| | S 200 μL | Citrate, pH | Citrate, | pH 4.0, 50 mM | Citrate, pH | Tris, pH |
| | | 4.0, 50 mM | pH 4.0, 50 | NaCl, 0.005% | 6.0, | 7.0, 1M |
| | | NaCl, | mM | w/v PS-80, 5 | 0.005% | NaCl, |
| | | 0.005% w/v | NaCl, | CVs | w/v PS-80 | 0.005% |
| | | PS-80, 9 | 0.005% | | 50 to 1500 | w/v PS- |
| | | CVs | w/v PS- | | mM NaCl | 80, 5 |
| | | | 80, 30 | | in 24 CVs | CVs |
| | | | CVs | | | |
| 12 | Nuvia ™ | 50 mM Tris, | 70 mM | 50 mM Tris, | 50 mM | 100 mM |
| | HP-Q | pH 9.0, 50 | Tris, pH | pH 9.0, 50 mM | Tris, pH | Tris, pH |
| | 200 μL | mM NaCl, | 9.0, 50 | NaCl, 0.005% | 9.0, | 7.0, 1M |
| | | 0.005% w/v | mM | w/v PS-80, 5 | 0.005% | NaCl, |
| | | PS-80, 9 | NaCl, | CVs | w/v PS-80 | 0.005% |
| | | CVs | 0.005% | | 50 to 1000 | w/v PS- |
| | | | w/v PS- | | mM NaCl | 80, 5 |
| | | | 80, 30 | | in 16 CVs | CVs |
| | | | CVs | | followed | |
| | | | | | by a step | |
| | | | | | at 50 mM | |
| | | | | | Citrate, pH | |
| | | | | | 9.0, 1000 | |
| | | | | | mM NaCl, | |
| | | | | | 0.005% | |
| | | | | | w/v PS-80 | |
| | | | | | for 3 CVs | |

### Column Challenge Studies

The aforementioned robotic system and methodology were also employed to perform column challenge experiments. These were carried out by increasing the levels of impurities presented to the chromatography column and observing how well full mature virus particles could be separated from impurities such as host cell DNA and bovine serum albumin (BSA). For this purpose, 0.6 mL Poros™ 50 HS columns were equilibrated for 5 CVs before they were loaded for 20 CVs and washed for 5 CVs with equilibration buffer. The columns were then eluted for 13 CVs with a slope of 75 mM CV⁻¹ and stripped for 5 CVs. Fractions were collected every 200 μL in UV transparent 96 well microplates (Corning Inc.) and the residence time was set to 2 min across all steps. The employed mobile phases during the equilibration and wash steps were comprised of a 50 mM citrate, 100 mM NaCl, 0.005% w/v PS-80 buffer system at different pH values. These spanned a pH range of 3.8-4.2 and remained constant across the entire separation. To generate buffers employed during the elution of the columns, the equilibration and wash buffers were mixed at desired ratios with a 50 mM citrate, 1000 mM NaCl, 0.005% PS-80 buffer prepared at the same pH. The latter was also used to strip the columns. Finally, here the load to the columns was the product from an early application of the preceding Affinity Chromatography step diluted 3-fold in concentrated mobile phase to match the equilibration buffer composition. Post dilution, the load was spiked with BSA (Sigma-Aldrich, MO, USA) and λ DNA (ThermoFisher Scientific) to concentrations of 0.1 g L⁻¹ and 200 ng mL⁻¹ respectively. These corresponded to loading to the columns amounts of 1.2 mg BSA and 2.4 μg λ DNA which represented a ˜>100-fold increase of such impurities in a typical Affinity Chromatography product. Fractions and their pools were analyzed via analytical methods including quantitative western blotting, SDS-PAGE, Quant-iT™ PicoGreen™ dsDNA (Invitrogen, CA, USA) and Pierce™ Coomassie Plus (Bradford) total protein assay (ThermoFisher Scientific).

### Batch 96 Well Plate Chromatography Method Description

Chromatography experiments in batch mode were performed using 96 well PreDictor™ chromatography plates pre-dispensed with 20 μL of resin Capto™ S ImpAct (both from Cytiva, MA, USA). The plates were operated manually and based on the manufacturer's instructions. When deviations from the suggested protocol were employed, these are detailed in the description of the derived results.

### Quantitative Western Blotting, SDS-PAGE and Colorimetric Analytical Methods

Before describing the analytical methods, it is important to first underline a key process in the morphogenesis of enteroviruses, CVA21, with a detailed review provided in [8] and depicted here schematically in FIG. 1. The capsids of full mature virus particles for enteroviruses are composed of 60 copies of four viral polypeptides (VP) VP1-VP4 arranged in a shell that packages the RNA genome. The generation of such full mature virus particles is the result of a complex morphogenesis comprised of seven steps. This includes the formation of empty procapsids that are composed of 12 pentamers of VP0, VP1 and, VP3, which is followed in a final step by the generation of full mature virus particles through the autocatalytic cleavage of VP0 into VP2 and VP4. This results in 12 pentamers of VP4, VP2, VP3 and VP1 encapsidating the RNA genome. Hence, empty procapsids are composed of proteins VP0, VP3 and VP1 whereas full mature virus particles are composed of proteins VP4, VP2, VP3 and VP1. Consequently, the assays described below aim to track full mature virus particles and empty procapsids via quantifying or visualizing VP2 and VP4 in the former case and VP0 in the latter case.

### Quantitative Western Blotting for Analysis of VP0 and VP4

Starting material, fractions and elution pools were assayed for full mature virus particles (VP4) and empty procapsids (VP0) via quantitative western blotting using a Sally Sue™ system and a 12-230 kDa Sally Sue™ Separation Module kit (Protein Simple, CA, USA). Samples were prepared using an Anti-Rabbit Detection Module (Protein Simple), according to the manufacturer's protocol, and denatured in a Mastercycler® Gradient (Eppendorf, NY, USA) for 5 min at 95° C. For their analysis, an anti-VP4 rabbit pAb (Lifetein LLC, NJ, USA) was used which was diluted to 20 μg mL⁻¹ in Antibody Diluent 2 (Protein Simple). Upon their preparation, the samples were loaded to the capillaries for 9 sec, separated for 40 min at 250 V, and immobilized for 250 sec. This was followed by their exposure to antibody diluent for 23 min, to anti-VP4 rabbit primary antibody for 30 min, and to the anti-rabbit secondary antibody for 30 min. The capillaries were then imaged with the chemiluminescence detection settings and the HDR detection profile. For data analysis purposes, the results were analyzed using the 8 sec exposure time setting with a dropped lines method for peak integration. All samples were diluted with a concentrated Tris, pH 7.5 buffer, 0.005% w/v PS-80 to a final composition of ˜150 mM Tris, pH 7.5, 0.005% w/v PS-80 prior to their analysis.

### SDS-PAGE Analysis for Visualization and Confirmation of VP0 and VP2

Fractions, pools and starting materials were also analyzed via gel electrophoresis using NuPAGE™ 12% Bis-Tris 1.0 mm 10-well gels (Invitrogen, CA, USA) to track empty procapsids and full mature virus particles (VP0 and VP2 respectively—VP4 has a molecular weight close to the low limit of the gel and cannot be reliably tracked). For this purpose, 700 μL of denaturing buffer was prepared by mixing 200 μL of NuPAGE Sample Reducing Agent (10×) (Invitrogen) and 500 μL of NuPAGE LDS Sample Buffer (4×) (Invitrogen). 14 μL and 26 μL per well of denaturing buffer and sample, respectively, were mixed together in a 96 well PCR plate (ThermoFisher Scientific) which was then sealed with an adhesive aluminum foil and centrifuged for a few minutes at 3000 rpm on a Sorvall™ Legend™ XTR centrifuge (ThermoFisher Scientific). The PCR plate was then denatured in a Mastercycler Gradient (Eppendorf) for 10 min at 70° C. Following denaturation, 25 μL of sample per lane were loaded into separate lanes of a gel with the latter also including a lane loaded with 2 μL of Mark12 Unstained Standard (Invitrogen). The prepared gels were electrophoresed in a 1×MOPS running buffer, prepared from NuPAGE MOPS SDS Running Buffer (20×) (Invitrogen), for 50 min at 200 V. The gels were then stained with a Pierce™ Silver Stain Kit (ThermoFisher Scientific) according to the manufacturer's protocol, with a 2 min development time. Finally, the gels were imaged with a Gel Doc™ EZ System (Bio-Rad) with a Silver Stain autoexposure scan protocol.

### Virus Purification by Sucrose Density Gradient

Starting material and collected fractions used for and generated from the cation exchange chromatography experiments respectively were analyzed for verification of presence of empty procapsids and full mature virus particles via sucrose density gradient centrifugation. For this purpose, four linear gradients were prepared at 11 mL using buffers of 15 mM Tris, 150 mM NaCl, 0.005% w/v PS-80, pH 8.0 containing sucrose levels at concentrations of 45% (w/v) and 15% (w/v). Upon their preparation, 1 mL of samples were layered on their top and the gradients were centrifuged at 36000 rpm for 100 min at 4° C. using an Optima™-SE Ultracentrifuge (Beckman Coulter, CA, USA). Post centrifugation, twelve fractions of equal volumes were collected from the top of the gradients and stored at 4° C. until further processing.

### Total Protein, DNA, and BSA Analytics

Colorimetric assays Quant-iT™ PicoGreen™ dsDNA (Invitrogen, CA, USA) and Pierce™ Coomassie Plus (Bradford) (ThermoFisher Scientific) were deployed as per the manufacturer's instructions. The aforementioned SDS-PAGE protocol was also used to track BSA in the assayed fractions and pools. BSA tracking was also performed via quantitative western blotting as described in [18].

### Example 2: Separation of Empty Procapsids and Full Mature Virus Particles with Ion Exchange Chromatography

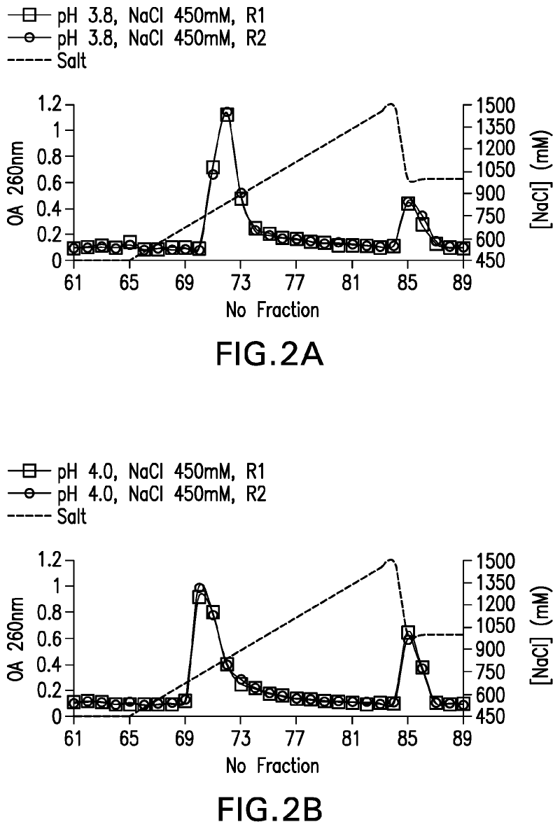

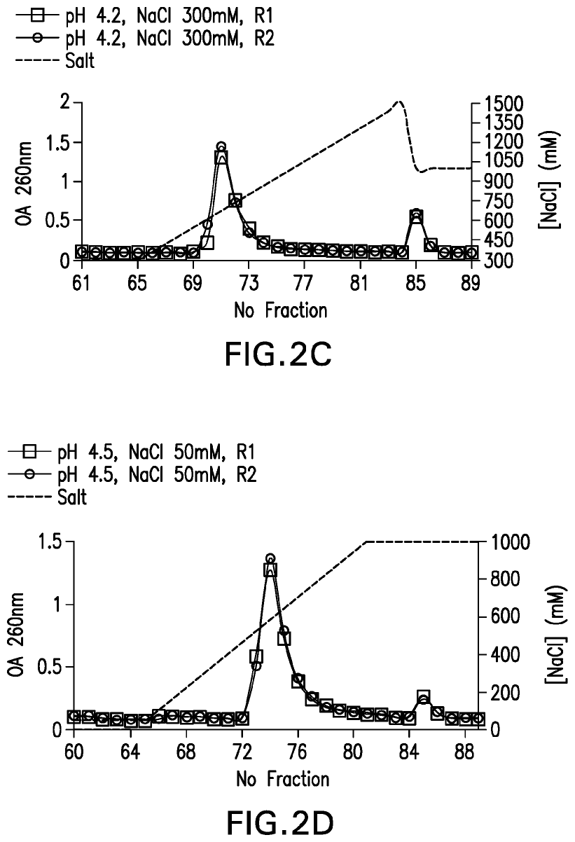

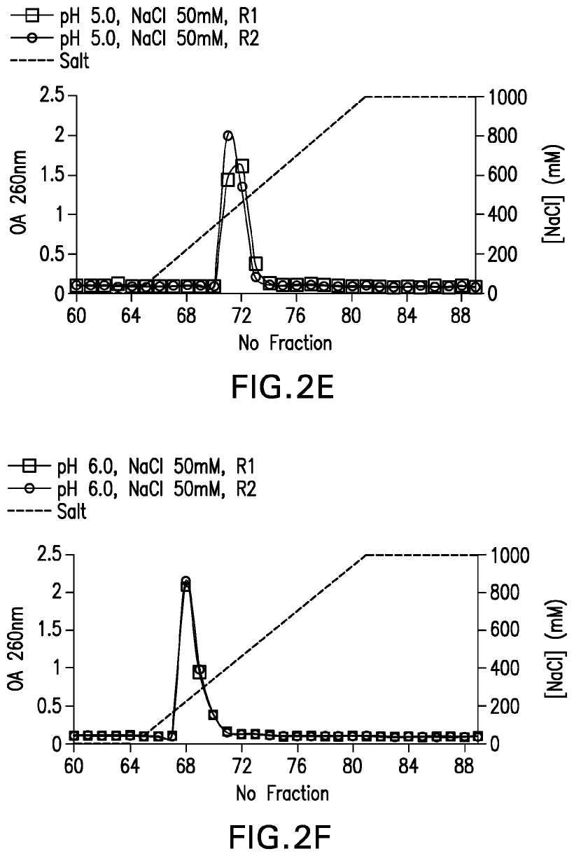

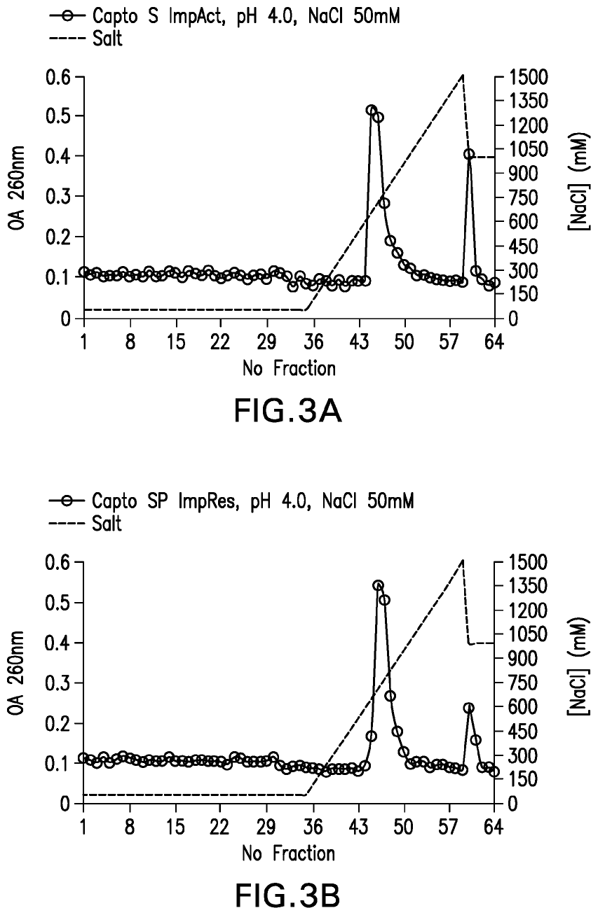

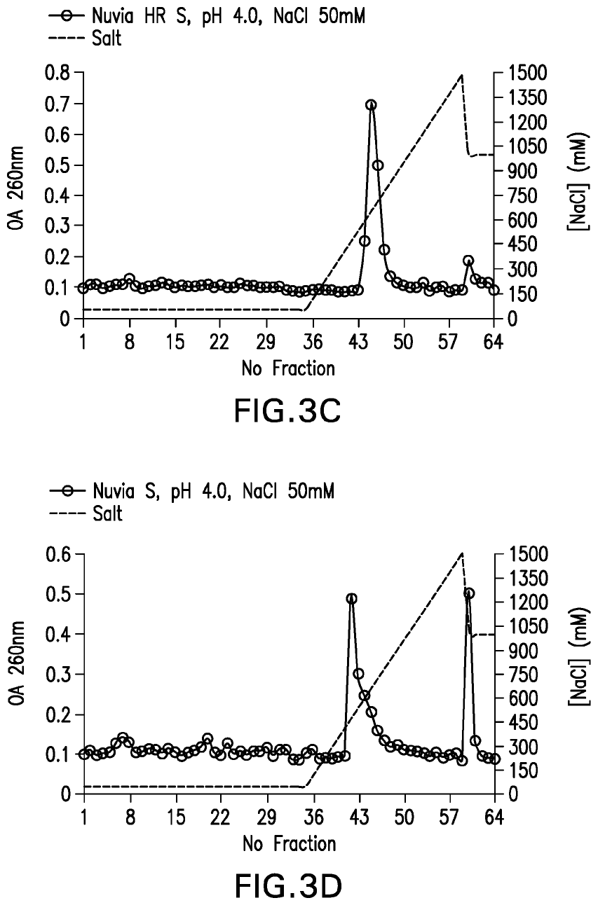

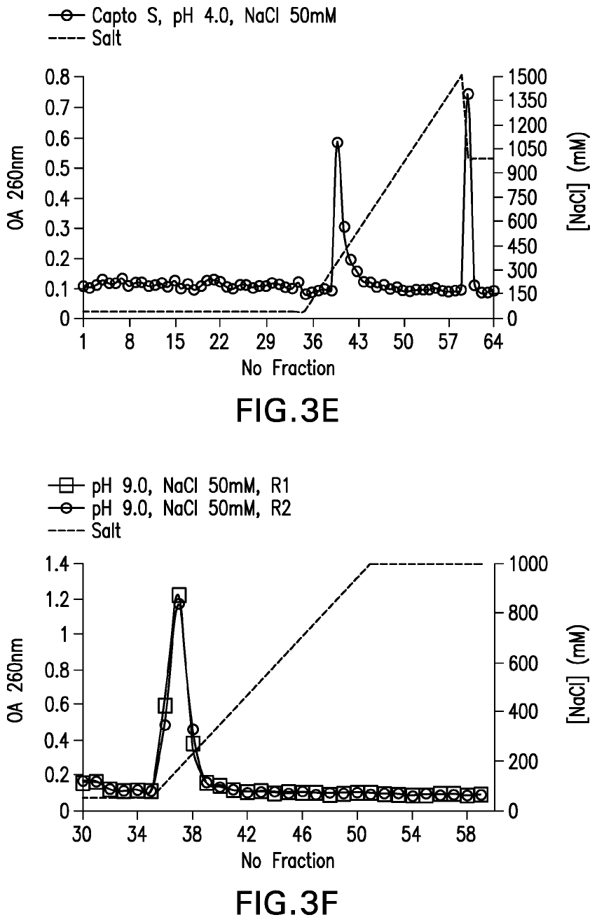

The chromatographic traces recorded across the performed separations showed the excellent repeatability of the RoboColumn method since the duplicated runs yielded traces overlapping with each other almost perfectly (FIG. 2). Based on the traces from the absorbance at 260 nm, the separations employing the CEX resins showed little to no breakthrough, (FIG. 3), whereas for the AEX resin Nuvia HP-Q a weak breakthrough was observed from the early stages of the column load.

### Cation Exchange Chromatography—Poros™ 50 HS Resin in Bind and Elute Mode

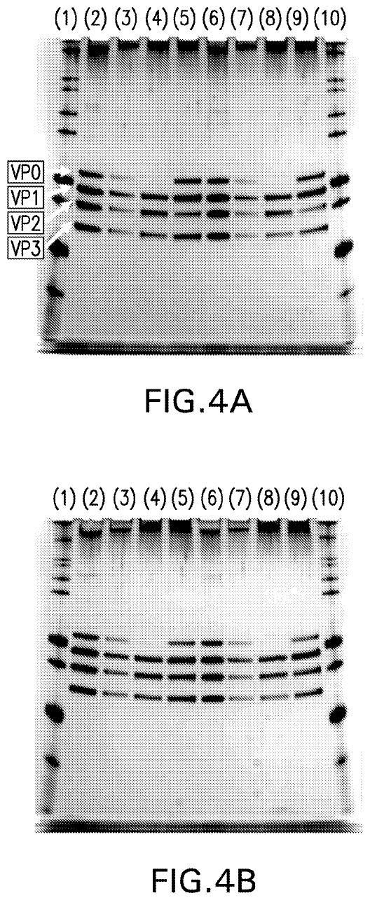

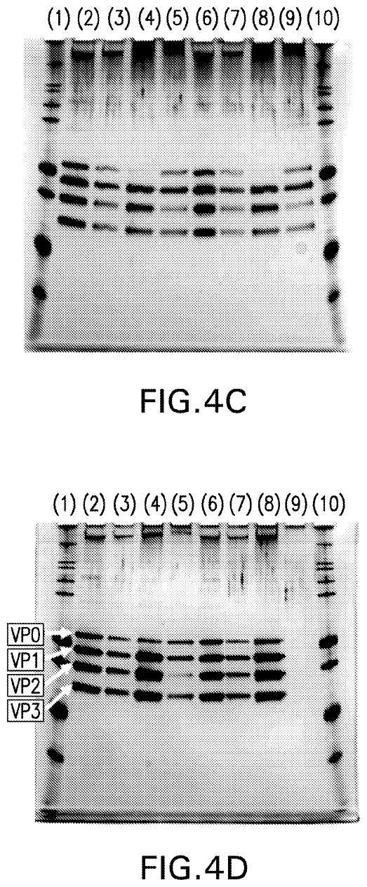

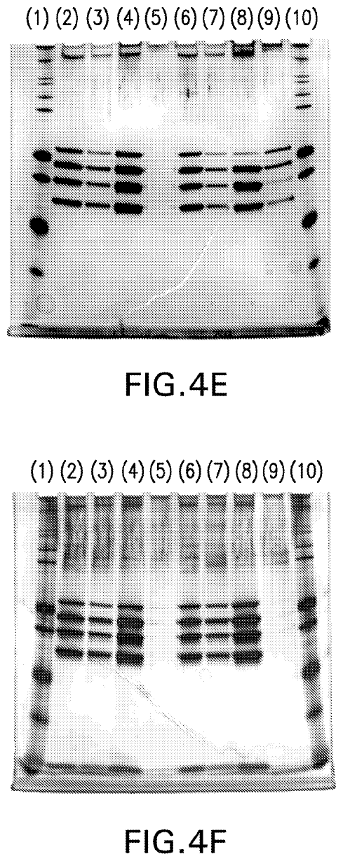

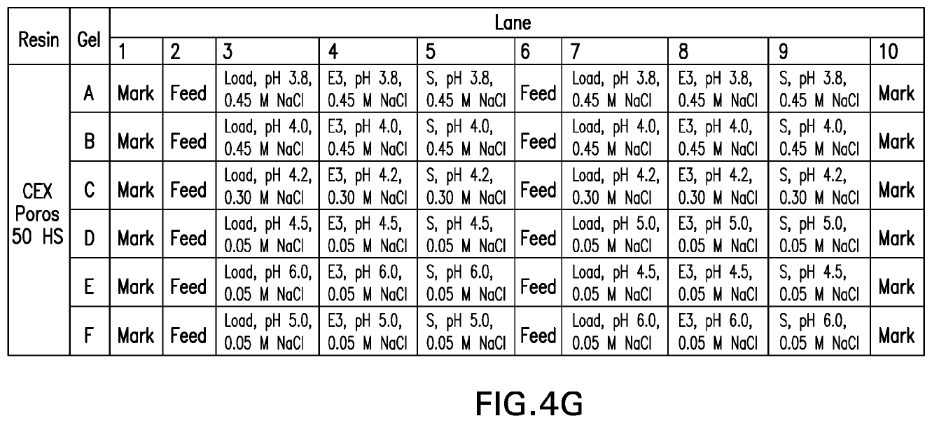

A single strong peak was observed in the elution gradient for all separations (FIGS. 2 and 3). For the CEX resins a second peak was also observed (FIGS. 2A-F and FIGS. 3A-E) in the strip with an area decreasing as the pH increased as indicated by the results for Poros™ HS 50 (FIGS. 2A-F). For example, at pH 5.0 and 6.0 (FIGS. 2E and 2F respectively) no peak was observed in the strip whereas at a pH 4.5 (FIG. 2D) a small peak was present which increased drastically at a pH of 3.8 (FIG. 2A). This suggested the presence of two populations of solutes, a strongly binding one, eluting in the strip, and a population with weaker retention eluting in the salt gradient. This was confirmed via SDS-PAGE analysis of elution pool E3 and the strip pool (S) (FIG. 4). This unexpected result became even more surprising when the analysis showed that at low pH values (e.g., pH of 3.8-4.5) the peak eluting in the strip included primarily empty procapsids (abundant VP0 band and little to no presence of VP2 band) whereas the peak eluting in the salt gradient contained primarily full mature virus particles (FIGS. 4A-D). Conversely, at high pH values (e.g., pH of 5.0 and 6.0 in FIGS. 4E and F respectively) the strip showed no presence of solutes, as expected since the chromatograms in FIGS. 2E and F also showed no peak, whereas the peak eluting in the salt gradient contained both empty procapsids and full mature virus particles. Hence, the chromatograms (FIG. 2) and SDS-PAGE analysis (FIG. 4) showed that the separation between empty procapsids and full mature virus particles was strongly dependent on the pH for resin Poros™ 50 HS; At low pH values (e.g., pH of 3.8-4.5), the empty procapsids were strongly retained on the resin and eluted only when the mobile phase had both a neutral pH and high salt content whereas the full mature virus particles eluted in the salt gradient. As the pH increased, the retention of both the full mature virus particles and empty procapsids decreased and they both co-eluted in the salt gradient.

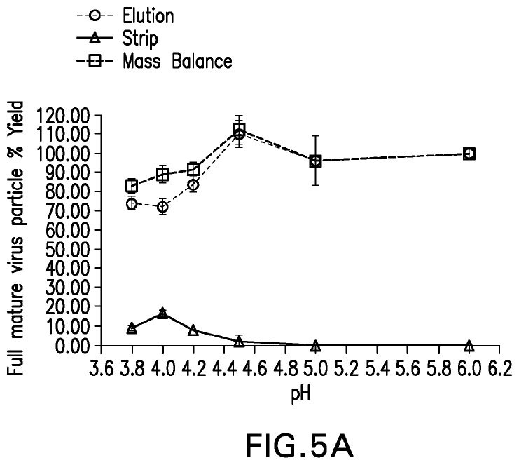

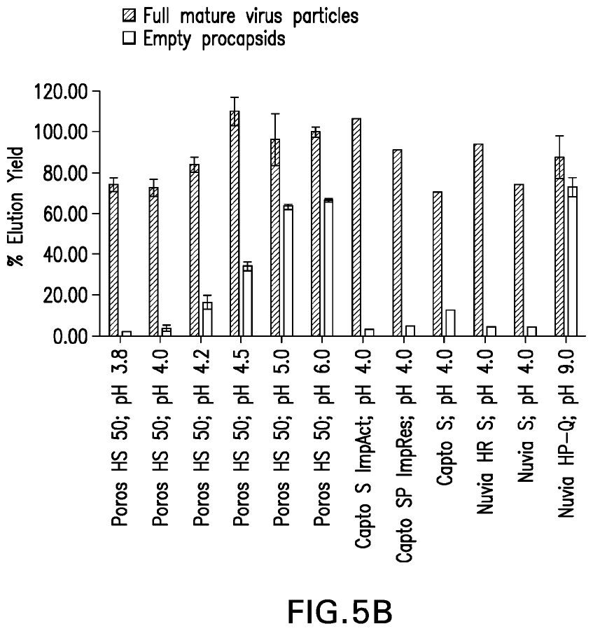

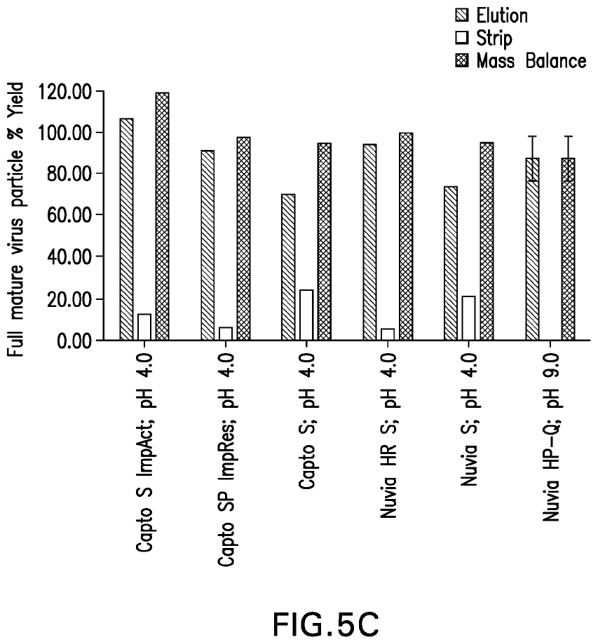

These results indicate that the empty procapsids are more retained than the full mature virus particles under low pH value conditions. This can be observed in the SDS-PAGE results since at low pH values (e.g., FIG. 4A), while the pool E3 contains no VP0 bands, a VP2 band is present in the strip pool suggesting an incomplete elution of full mature virus particles in the salt gradient. This was supported further by quantitative western blotting results (FIG. 5A). Across all pH conditions, Poros™ 50 HS yielded mass balances of full mature virus particles in excess of 80% with their amount in elution pool E3 (i.e., elution yields) increasing with increasing pH (˜75%-˜100%). Conversely, their amount in the strip decreased with increasing pH.

While the elution yields of the full mature virus particles undergo an increase from ˜75% to 100% by increasing the pH (FIG. 5A), the impact of increasing the pH on the elution yields of the empty procapsids is considerably more significant (FIG. 5B). Here, at pH values of 4.2 and below, elution yields up to ˜15% are observed which increase to ˜60% at a pH of 5.0 and above (FIG. 5B) leading to significant mixing between empty procapsids and full mature virus particles. As a result, the retention of empty procapsids on the CEX resin Poros™ 50 HS is considerably more sensitive to pH than the retention for the full mature virus particles.

### Conditions for Separating Full Mature Virus Particles and Empty Procapsids in Bind and Elute Mode

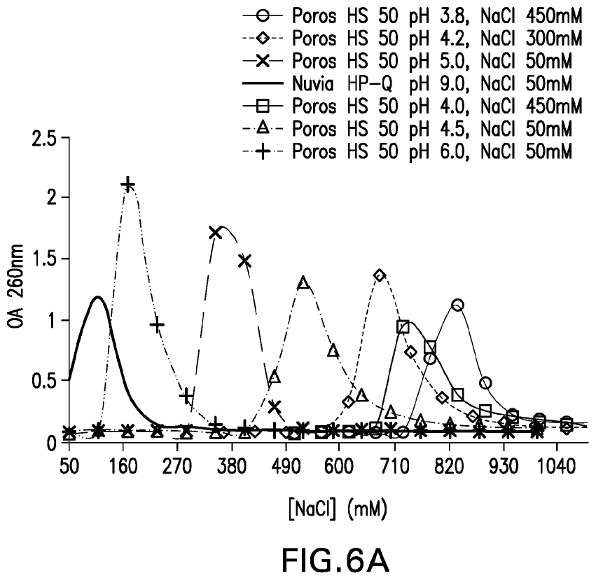

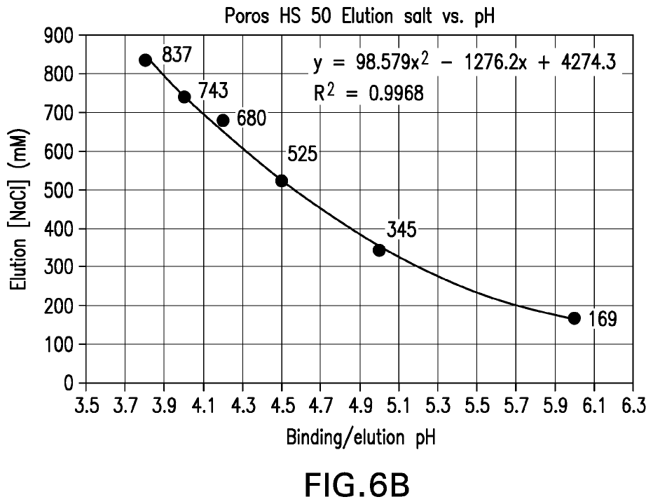

For the CEX step to be successful in separating full mature virus particles and empty procapsids it must also lead to high elution yields while minimizing any yield losses due to full mature virus particles flowing through during the loading of the column. As mentioned, across all tested conditions for Poros™ 50 HS, and for the rest of the CEX resins, the quantitative western results showed no full mature virus particles or empty procapsids in the flowthrough and wash fractions. Hence, all performed separations showed 100% binding yields even if the chromatography traces showed weak flowthrough signals at 260 nm (i.e., impurities flowing through and full mature virus particles or empty procapsids). The high binding yields for Poros™ HS were also accompanied by wide operating windows in terms of binding salt level as a function of the pH (i.e., salt level in equilibration, load and wash phases and also at the beginning of the salt gradient). FIG. 6 shows that the elution of the main peak, increasingly comprised primarily of full mature virus particles as the pH decreases (FIG. 5B), takes place at high salt levels, even at a pH of 5.0. Hence, during the loading of the Poros™ 50 HS column the binding salt could vary within a wide range without a negative impact on binding yields. As result, this step shows excellent robustness in terms of binding salt levels and in particular at pH levels up to 4.5 wherein a significant separation between empty procapsids and full mature virus particles can be achieved.

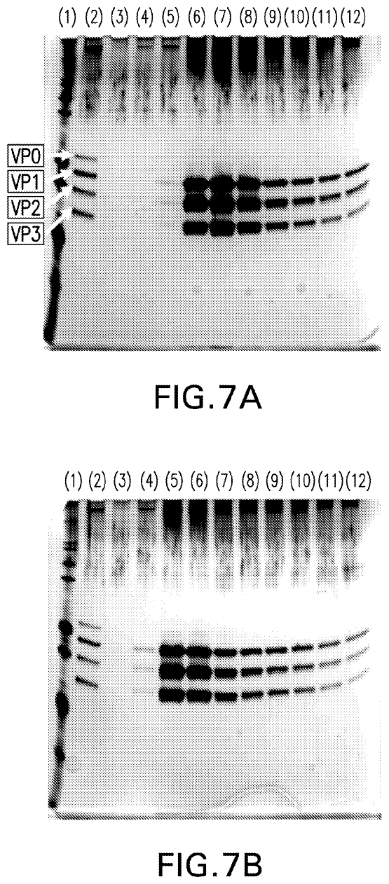

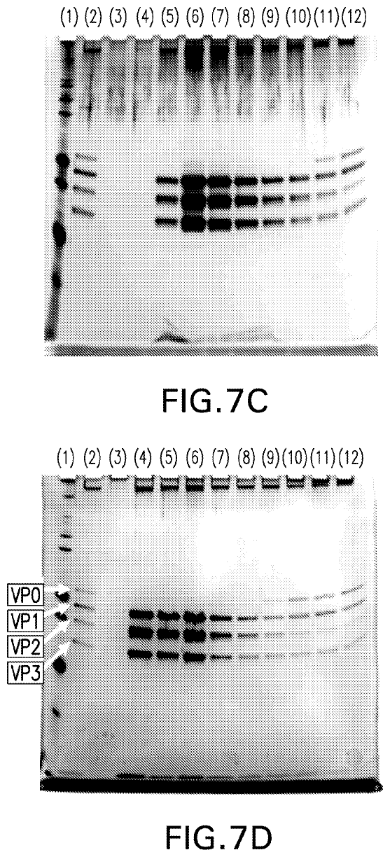

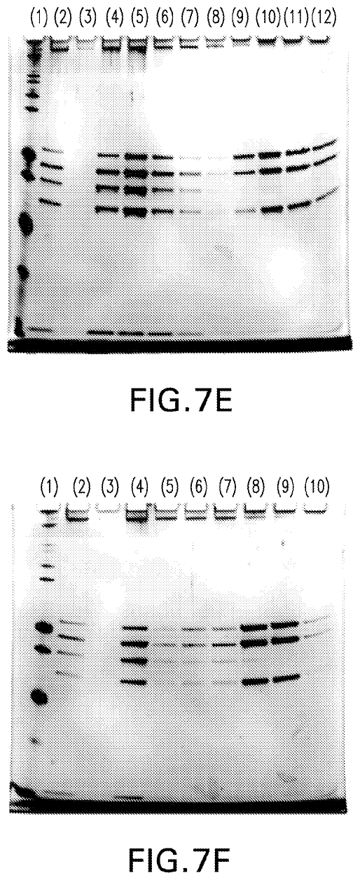

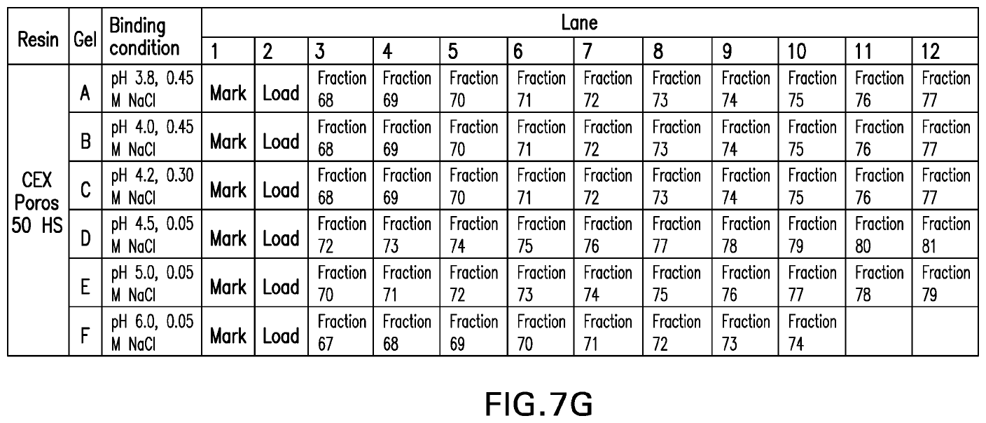

The elution yields for the full mature virus particles and empty procapsids in FIGS. 5A and 4B suggest that the CEX step can be performed at a pH of 4.0 for complete separation between empty procapsids and full mature virus particles. At pH 4.0 high elution yields (˜75%) can be achieved while the elution pool (E3) shows no presence of empty procapsids (FIG. 4B). At such conditions, the empty procapsids elute in the strip along with a small amount of full mature virus particles (˜15%). While FIG. 5B and the gels in FIG. 4 suggest that small deviations in pH can result in a significant change in the relative abundance of full mature virus particles in the elution pool, this is not accurate. The aforementioned figures depict results based on elution pool E3. This contains the entire main elution peak and is extended further to contain fractions at higher salt levels following its elution (i.e., it is wider than needed). SDS-PAGE analysis of individual fractions (FIG. 7) shows how the full mature virus particles and empty procapsids are separated within the salt gradient and within fractions also contained in pool E3. FIGS. 7A and B show that there is no mixing of empty procapsids and full mature virus particles (i.e., no VP0 bands) at pH of 3.8 and 4.0 across the gradient as suggested by FIGS. 4A, B and 5B. However, FIGS. 7C and 7D, corresponding to pH of 4.2 and 4.5, show a separation of full mature virus particles and empty procapsids within the salt gradient. At pH 4.2 fractions 70-75 contain ˜90% of the eluted full mature virus particles (FIG. 7C) whereas at a pH of 4.5 fractions 73-77 contain ˜85% of the eluted full mature virus particles (FIG. 7D). In both cases these fractions show no presence of empty procapsids. For these two conditions, pool E3 contained fractions 68-84 and 71-81 respectively and therefore contained fractions at high elution salt levels that contained low amounts of full mature virus particles and were richer in empty procapsids than full mature virus particles. Hence, a careful selection of fractions to include in the product pool, by excluding the tail of the main elution peak, would eliminate any mixing between empty procapsids and full mature virus particles and lead to high elution yields even when the pH is as high as 4.5 (elution yields of full mature virus particles of ˜76% and ˜85% at pH 4.2 and 4.5 respectively). This however is not possible for pH of 5.0 and above since empty procapsids and full mature virus particles co-elute across the gradient (FIGS. 7E and 7F).

Hence, taking into consideration the elution yields from elution pool E3 (FIGS. 5A and B), the retention trends as function of pH (FIG. 6) and the separation between the full mature virus particles and empty procapsids in the gradient (FIG. 7) it is concluded that CEX resin Poros™ 50 HS, run in bind and elute mode, can yield a robust separation of full mature virus particles and empty procapsids and with high yields of full mature virus particles. Other CEX resins yield similar separation results as discussed in Example 4. Binding conditions with a pH between 3.8 and 6.0 and a NaCl concentration decreasing with increasing pH between 50 mM and 600 mM can be used to load the column. Such conditions ensure that all full mature virus particles bind to the column at process relevant loading challenges. The column can then be washed with a mobile phase condition matching the binding condition before it is eluted. For the elution of the column, a condition can be used with a pH between 3.8 and 4.5, and a NaCl concentration decreasing with increasing pH between 550 mM and 850 mM. This will ensure that full mature virus particles are eluted with high yields while empty procapsids remain bound to the column and eluted during its stripping with a neutral pH, high salt mobile phase condition.

Finally, an interesting and unexpected result of the CEX application at pHs of 5.0 and above needs to also be highlighted. The lane images in FIGS. 7E and 7F showed the presence of two populations of viral particles in the elution gradient. The particles eluting late in the gradient show viral bands for VP0, VP1 and VP3 with the lanes also showing evidence of RNA presence since it can be stained by silver stain. Conversely, the particles eluting in the early part of the gradient show viral bands for VP0, VP1, VP2 and VP3 and presence of RNA. Based on the known information on the morphogenesis of picornaviruses it is believed that particles eluting late in the CEX gradient at pHs of 5.0 and above are primarily comprised of pro-virions whereas the particles eluting early in the gradient are comprised of both empty procapsids and full mature virus particles. When CEX is performed at pHs below 5.0 (FIGS. 7A-7D) the earlier eluting particles are comprised only of full mature virus particles as there is no presence of VP0. This suggests that CEX can resolve full mature virus particles from any other type of viral particles when performed at a low pH conditions.

### Cation Exchange Chromatography—Poros™ 50 HS Resin in Flowthrough Mode

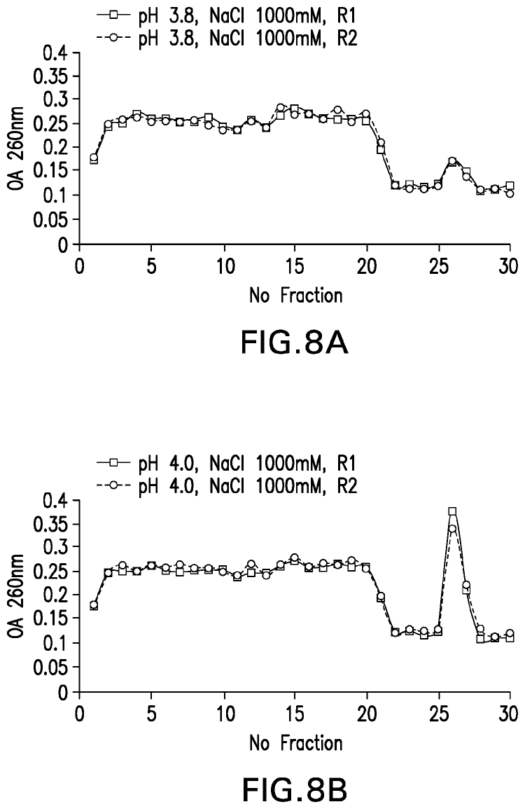

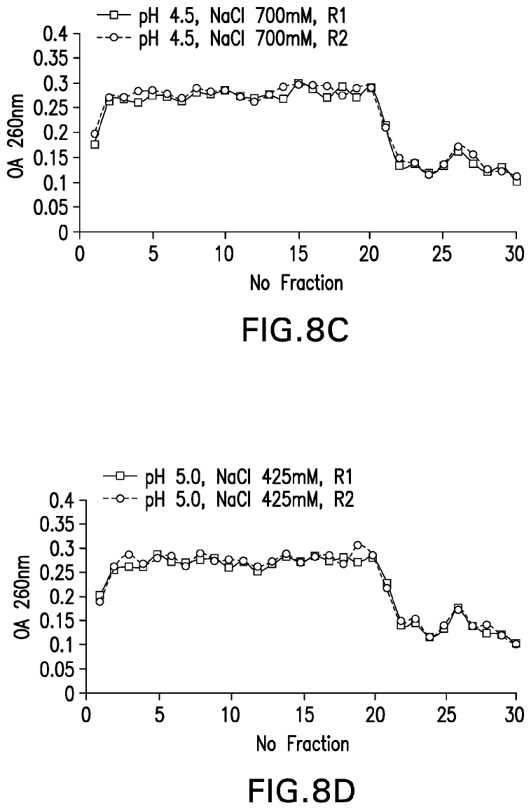

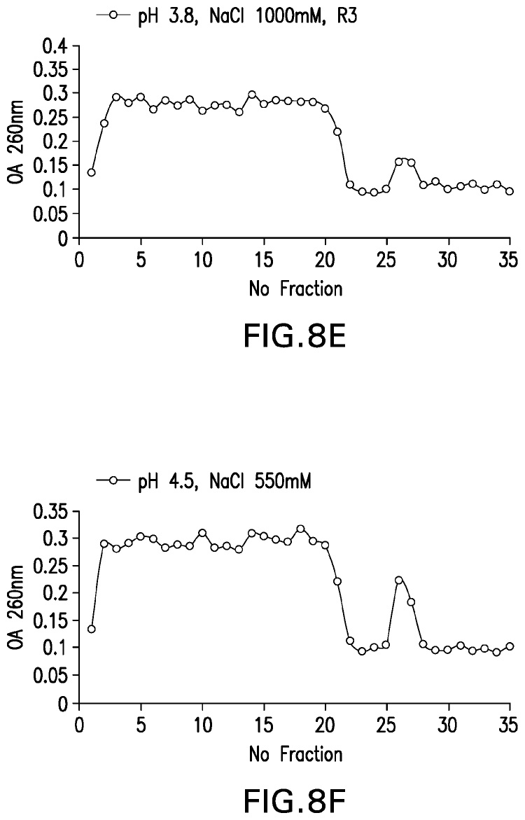

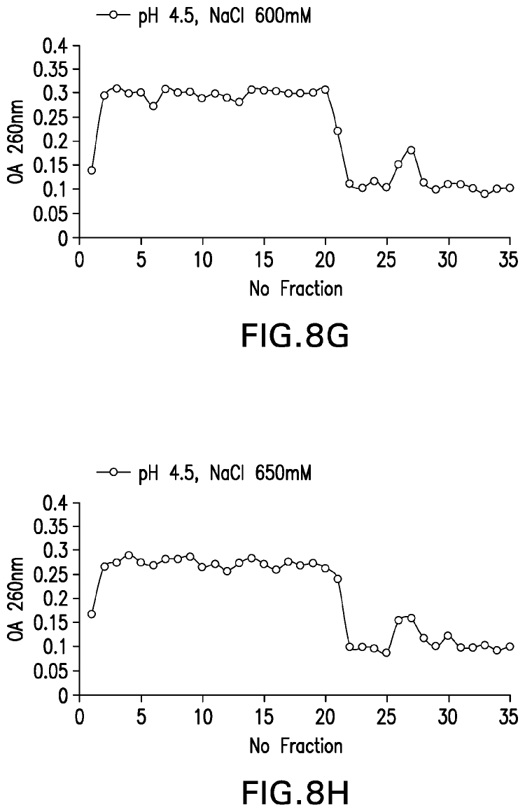

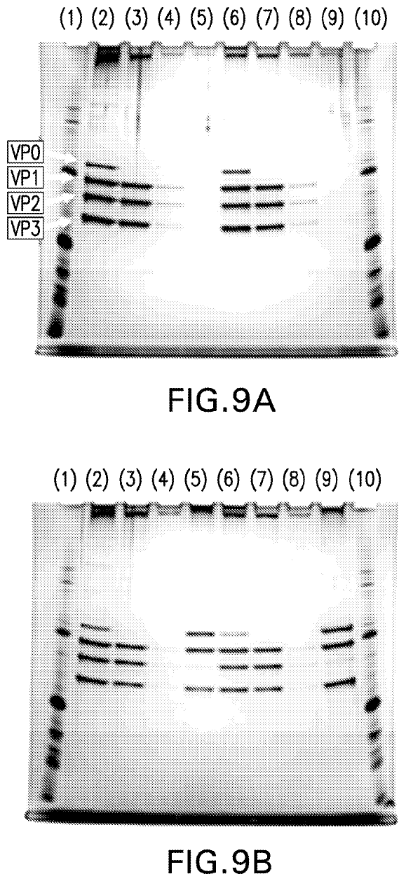

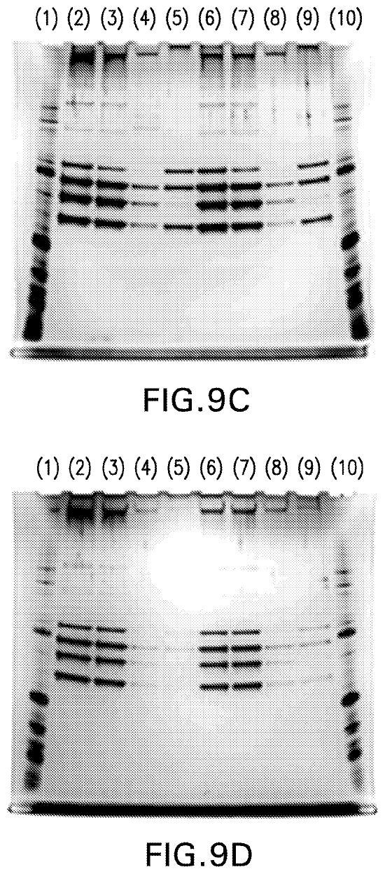

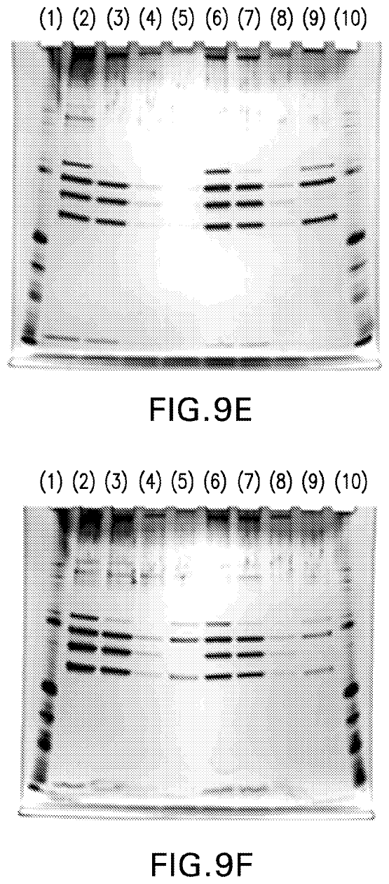

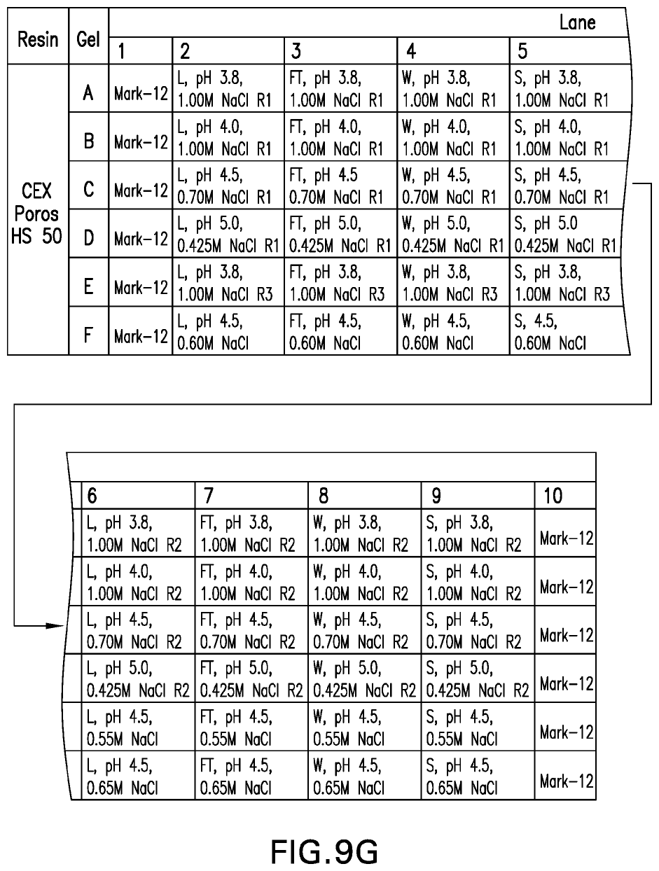

The retention trends in FIG. 6B, and in particular at pH values below 4.5, along with the SDS-PAGE results in FIG. 7 provided information that the full mature virus particles could be separated from empty procapsids by deploying the Poros HS 50 based CEX step in flowthrough mode. Here, full virus particles would flow through while empty procapsids would bind to the resin. Separations 13-24 were performed to test this and their details are shown in Table 3. Here, affinity chromatography product from upstream process B was adjusted to match the CEX equilibration conditions for each separation and was loaded to the columns for 20 CVs. Upon completion of the loading step, the columns were washed with equilibration buffer and then striped with a Tris buffer in one step (i.e., no step or gradient elution was performed). All steps were performed at a residence time of 2 min and the recorded chromatograms are shown in FIG. 8 whereas the SDS-PAGE analysis of the pooled fractions, collected during the loading (flowthrough), wash and strip phases, is shown in FIG. 9. The latter confirms that at pH values ≤4.5 (FIGS. 9A, B, C, E and F) the flowthrough fractions contain full mature virus particles with the content of empty procapsids being non-existent at pH 3.8-4.0 (FIGS. 9A, 9E and 9B respectively) and greatly reduced at pH 4.5 compared to the load (FIGS. 9C, E and F). This is further supported by the observation that for such pH values, the strip pool was shown to contain no VP2 (characteristic of full mature virus particles) while being rich in VP0 (characteristic of empty procapsids). Separation 20 (pH 5.0) showed no separation between empty procapsids and full mature virus particles in the flowthrough product (FIG. 9D).

TABLE 3
Details of chromatographic conditions screening the full mature virus particles/empty
procapsids separation in flowthrough mode on RoboColumns packed with 200 μL of
resin Poros ™ HS 50.

| Separation | Equilibration | Wash | Strip |
|---|---|---|---|
| 13 | 50 mM Citrate, 1000 | 50 mM Citrate, 1000 | 100 mM Tris, 1000 |
| | mM NaCl, 0.005% PS80, | mM NaCl, 0.005% PS80, | mM NaCl, 0.005% |
| | pH 3.8, 10 CVs | pH 3.8, 5 CVs | PS80, pH 7.0, 5 CVs |
| 14 | 50 mM Citrate, 1000 | 50 mM Citrate, 1000 | 100 mM Tris, 1000 |
| | mM NaCl, 0.005% PS80, | mM NaCl, 0.005% PS80, | mM NaCl, 0.005% |
| | pH 3.8, 10 CVs | pH 3.8, 5 CVs | PS80, pH 7.0, 5 CVs |
| 15 | 50 mM Citrate, 1000 | 50 mM Citrate, 1000 | 100 mM Tris, 1000 |
| | mM NaCl, 0.005% PS80, | mM NaCl, 0.005% PS80, | mM NaCl, 0.005% |
| | pH 4.0, 10 CVs | pH 4.0, 5 CVs | PS80, pH 7.0, 5 CVs |
| 16 | 50 mM Citrate, 1000 | 50 mM Citrate, 1000 | 100 mM Tris, 1000 |
| | mM NaCl, 0.005% PS80, | mM NaCl, 0.005% PS80, | mM NaCl, 0.005% |
| | pH 4.0, 10 CVs | pH 4.0, 5 CVs | PS80, pH 7.0, 5 CVs |
| 17 | 50 mM Citrate, 700 mM | 50 mM Citrate, 700 mM | 100 mM Tris, 1000 |
| | NaCl, 0.005% PS80, pH | NaCl, 0.005% PS80, pH | mM NaCl, 0.005% |
| | 4.5, 10 CVs | 4.5, 5 CVs | PS80, pH 7.0, 5 CVs |
| 18 | 50 mM Citrate, 700 mM | 50 mM Citrate, 700 mM | 100 mM Tris, 1000 |
| | NaCl, 0.005% PS80, pH | NaCl, 0.005% PS80, pH | mM NaCl, 0.005% |
| | 4.5, 10 CVs | 4.5, 5 CVs | PS80, pH 7.0, 5 CVs |
| 19 | 50 mM Citrate, 425 mM | 50 mM Citrate, 425 mM | 100 mM Tris, 1000 |
| | NaCl, 0.005% PS80, pH | NaCl, 0.005% PS80, pH | mM NaCl, 0.005% |
| | 5.0, 10 CVs | 5.0, 5 CVs | PS80, pH 7.0, 5 CVs |
| 20 | 50 mM Citrate, 425 mM | 50 mM Citrate, 425 mM | 100 mM Tris, 1000 |
| | NaCl, 0.005% PS80, pH | NaCl, 0.005% PS80, pH | mM NaCl, 0.005% |
| | 5.0, 10 CVs | 5.0, 5 CVs | PS80, pH 7.0, 5 CVs |
| 21 | 50 mM Citrate, 1000 | 50 mM Citrate, 1000 | 50 mM Tris, 1500 mM |
| | mM NaCl, 0.005% PS80, | mM NaCl, 0.005% PS80, | NaCl, 0.005% PS80, |
| | pH 3.8, 10 CVs | pH 3.8, 5 CVs | pH 7.5, 10 CVs |
| 22 | 50 mM Citrate, 550 mM | 50 mM Citrate, 550 mM | 50 mM Tris, 1500 mM |
| | NaCl, 0.005% PS80, pH | NaCl, 0.005% PS80, pH | NaCl, 0.005% PS80, |
| | 4.5, 10 CVs | 4.5, 5 CVs | pH 7.5, 10 CVs |
| 23 | 50 mM Citrate, 600 mM | 50 mM Citrate, 600 mM | 50 mM Tris, 1500 mM |
| | NaCl, 0.005% PS80, pH | NaCl, 0.005% PS80, pH | NaCl, 0.005% PS80, |
| | 4.5, 10 CVs | 4.5, 5 CVs | pH 7.5, 10 CVs |
| 24 | 50 mM Citrate, 650 mM | 50 mM Citrate, 650 mM | 50 mM Tris, 1500 mM |
| | NaCl, 0.005% PS80, pH | NaCl, 0.005% PS80, pH | NaCl, 0.005% PS80, |
| | 4.5, 10 CVs | 4.5, 5 CVs | pH 7.5, 10 CVs |

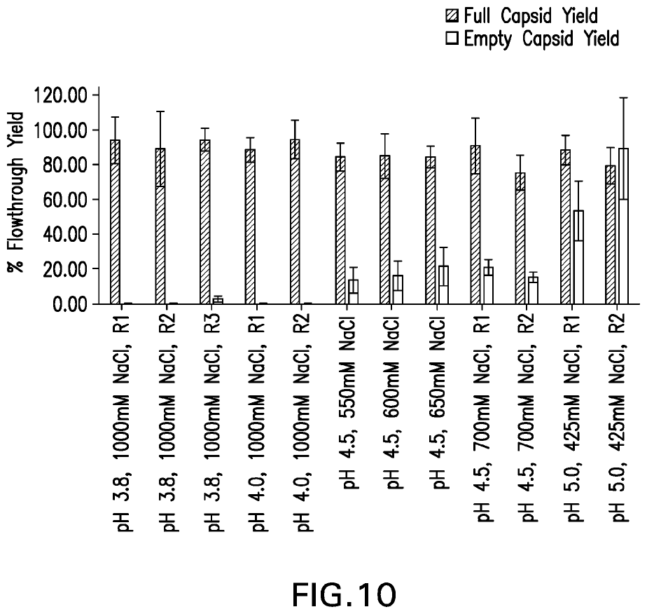

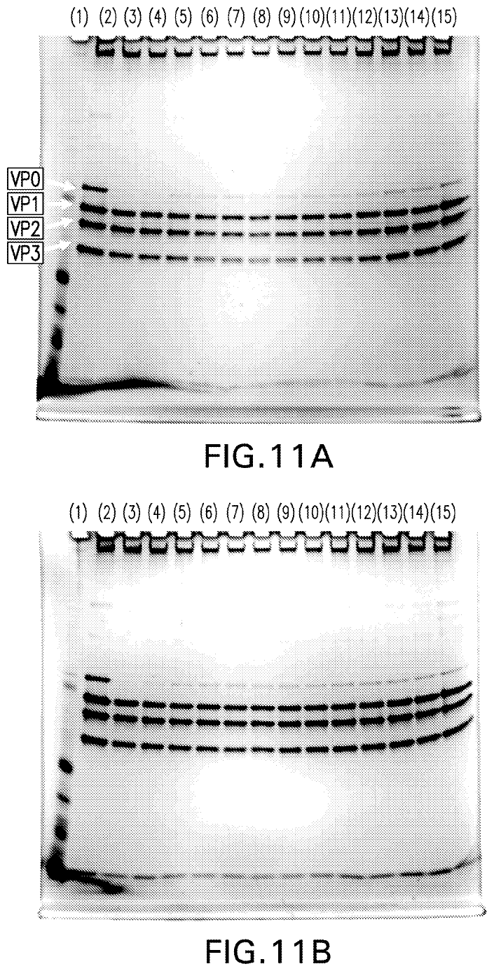

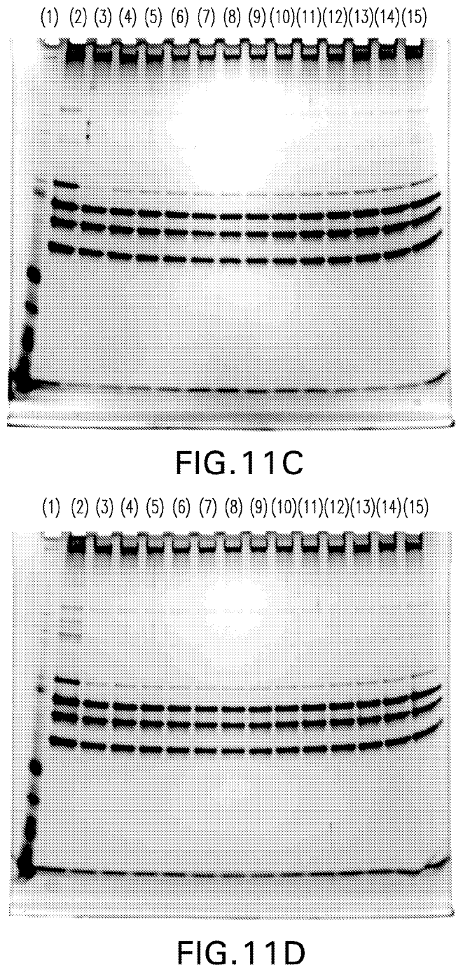

The achieved purification of full mature virus particles from empty procapsids renders the CEX step in flow through mode at low pH conditions as a viable alternative to running it in bind and elute mode. This was further supported by the resulting full mature virus particle yields (FIG. 10) which were found to be in excess of ˜80% and comparable to the bind and elute yields (FIG. 5B). The separation of the two particle populations is entirely robust at pH below 4.0. As FIG. 10 shows, and also depicted in the flow through pools in FIGS. 9C, 9E and 9F, at pH 4.5, a decrease in binding salt increases the amount of empty procapsids in the flow through product pool while also leading to potentially lower full mature virus particle yields. Hence, running the CEX step in flowthrough mode at pH 4.5 requires a trade-off between yield and purity (i.e., running at a lower salt level to bind fully empty procapsids while also binding a small amount of full mature virus particles). A small loss in yield due to this trade-off could also be balanced against the amount of affinity chromatography product loaded to the column; FIG. 11 shows that the empty procapsids break through slowly whereas the full mature virus particles flow through immediately. Consequently, a reduction in the amount loaded, along with a drop in the binding salt level, can lead to a flowthrough product pool free of empty procapsids and high yields even at a pH of 4.5.

### Example 3: Separation of Process Related Impurities from Full Mature Virus Particles

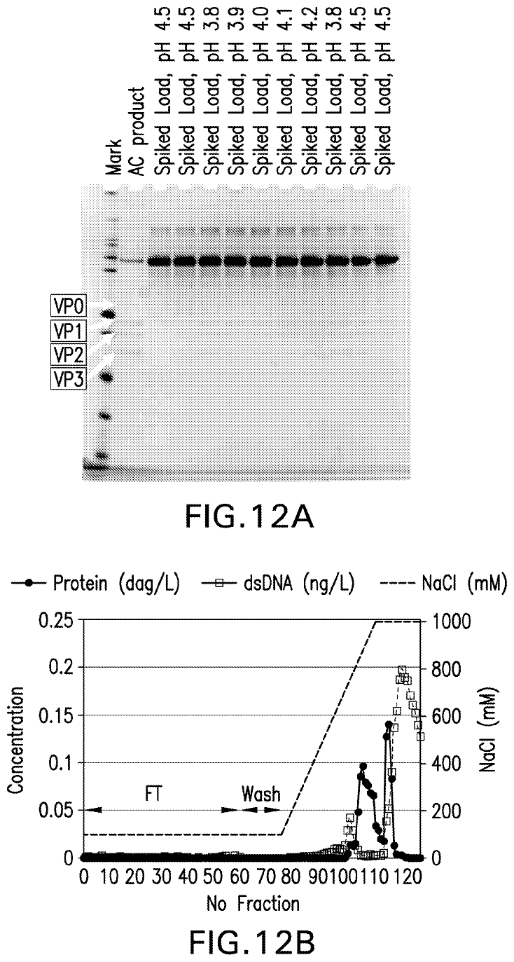

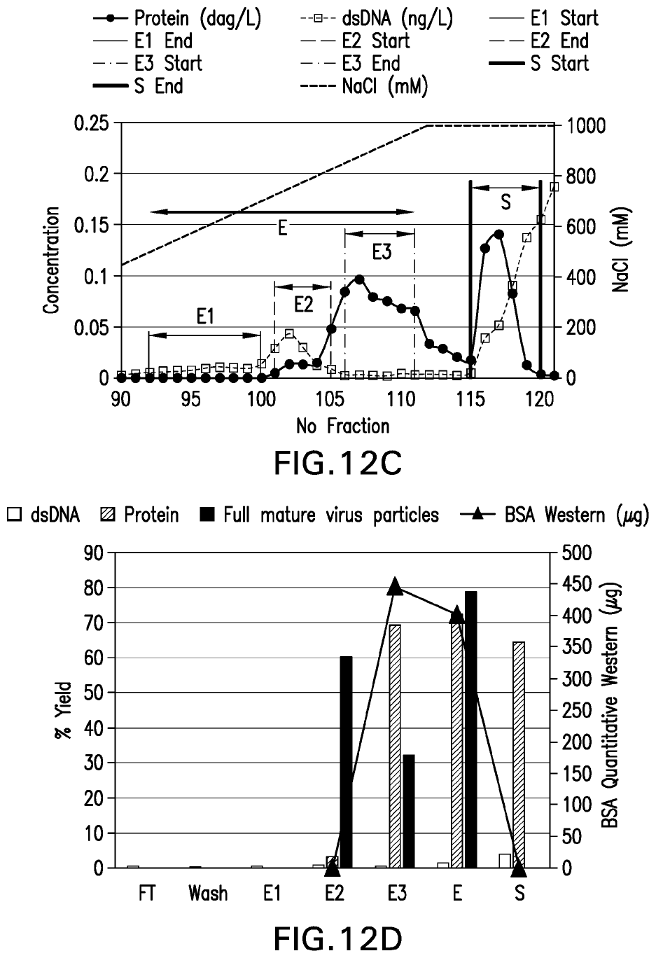

The CEX step in bind and elute mode is also capable of separating the full mature virus particles from impurities in the gradient and thus improving the purity of the elution product. The column challenge studies with BSA and DNA aimed to demonstrate this. FIG. 12A shows the considerably increased presence of impurities by comparing the elution product from the early application of the affinity chromatography step before and after the addition of the BSA and DNA spikes. At pH of 4.0, the elution profile from such a study (FIGS. 12B and 12C) showed two peaks (E2 and E3 in FIGS. 12C and 12D) being separated in the gradient. The earlier eluting one (E2 in FIGS. 12C and 12D) contained ˜60% of the loaded full mature virus particles with trace amounts of total protein content and BSA (FIG. 12D) whereas the second one (E3 in FIGS. 12C and 12D) contained ˜30% of the loaded full mature virus particles and large amounts of total protein and BSA (FIG. 12D). The chromatography trace obtained via the total protein assay (FIGS. 12B and 12C) showed that a large amount of proteins was still bound to the column even after the elution gradient. The PicoGreen assay (dsDNA) results showed one peak in the elution gradient (E2 in FIG. 12C) and a partially eluted peak in the strip (FIGS. 12B and 12C). The former coincided with the elution of the full mature virus particles and since all chromatograms reported here (FIGS. 2 and 3) showed a strong peak in the 260 nm, which was also rich in full mature virus particles, it is believed that it was tracking primarily the elution of the full mature virus particles. This was further supported by the fact that FIG. 12D showed that relative to the spiked amount of λ DNA only trace amounts of DNA were eluted across the entire gradient and strip while the mass balance for the full mature virus particles was closed to 90%. Hence, DNA was unexpectedly found to bind very strongly to the Poros™ 50 HS resin at pH 4.0 and is not eluted even at a NaCl level of 1 M. These results demonstrate that even in cases wherein the CEX step is challenged with considerably higher than expected amounts of proteinaceous and DNA impurities it can still result in a near baseline resolution between full mature virus particles and impurities.

### Example 4: Alternative Ion Exchange Media

### Cation Exchange Resins

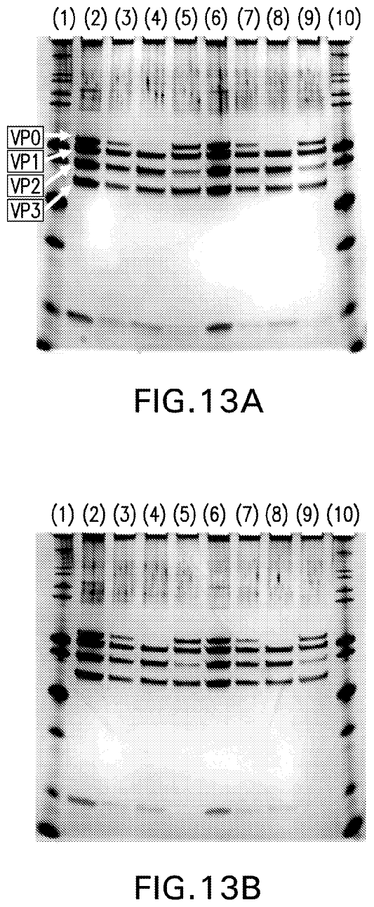

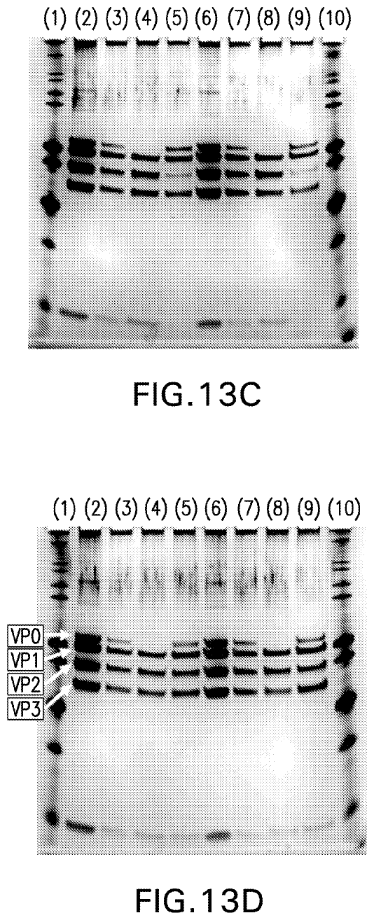

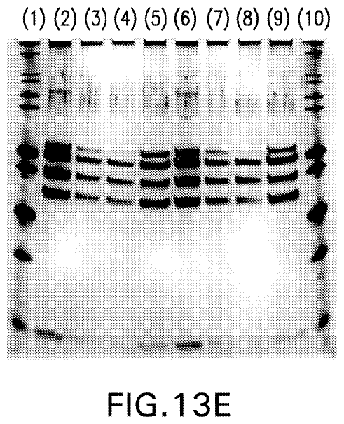

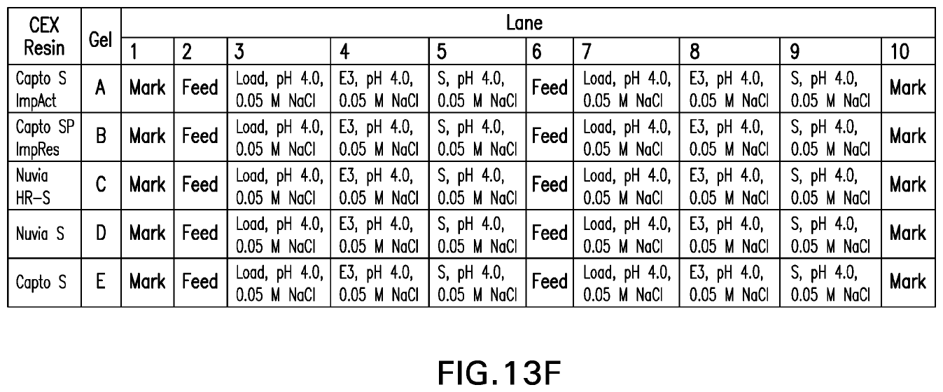

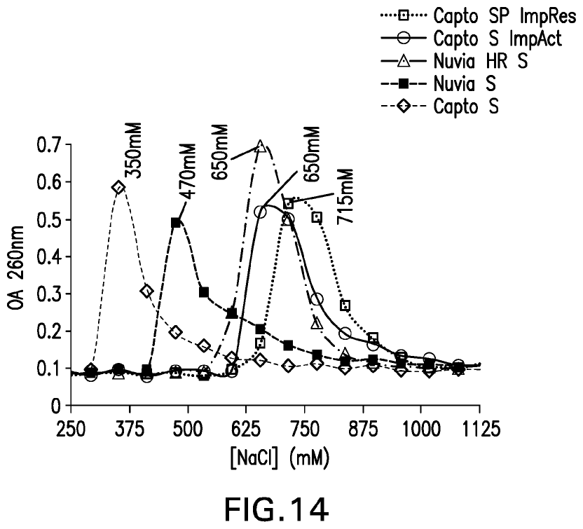

Five alternative cation exchangers were also tested, in addition to the cation exchange resin Poros™ 50 HS, to determine whether they could also deliver a good separation between empty procapsids and full mature virus particles at a pH of 4.0 (FIGS. 13A-E). SDS-PAGE analysis showed that all five resins separated full mature virus particles and empty procapsids with the latter being collected in the strip pool (i.e., no VP0 band in E3 pool) (FIG. 13). The VP0 elution yields, obtained based on quantitative western blotting, for the five resins agreed with the gel analysis since they were less than ˜10% (FIG. 5B).

### Anion Exchange Resin Nuvia HP-Q

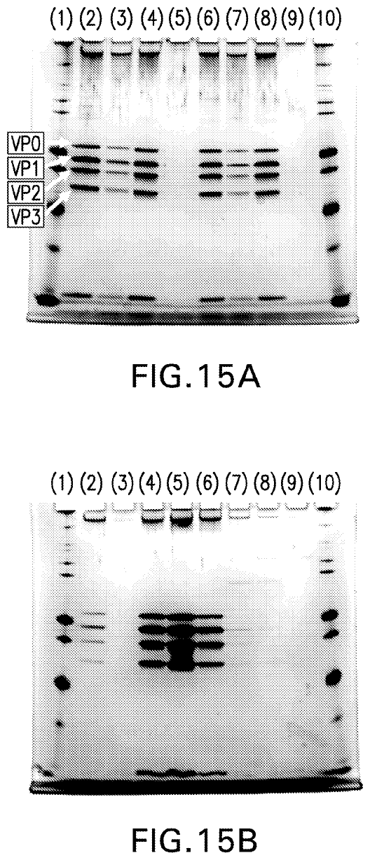

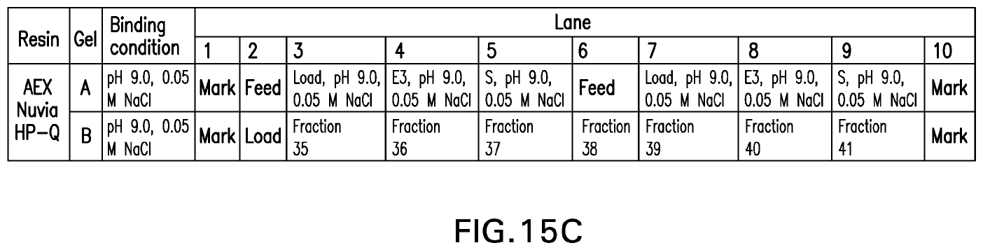

Conversely, the AEX resin Nuvia HP-Q, run at a pH of 9.0 (FIG. 3F), returned high elution yields but at the same time no resolution between empty procapsids and full mature virus particles, as shown by SDS-PAGE (FIG. 15) and quantitative western blotting (FIGS. 5B and C). Their retention on anion exchangers drops significantly as the pH decreases (for example both empty procapsids and full mature virus particles flow through at a pH of 8.5) which makes it impossible to derive an AEX method capable of both separating efficiently full mature virus particles and empty procapsids and leading to high yields of full mature virus particles.

An anion exchange resin, such as Nuvia HP-Q, run at strong binding conditions (no full mature virus particles or empty procapsids were detected in the flowthrough and wash fractions), cannot separate full mature virus particles and empty procapsids and hence lead to product pools with high yield and purity. In contrast, cation exchangers, evaluated in bind and elute mode, were characterized across a range of conditions and were shown to be able to deliver a robust step for separating full mature virus particles and empty procapsids viral while returning high elution yields for full mature virus particles. At the same time, the CEX step serves to concentrate the product, which facilitates further processing activities, while removing process impurities, which either flow through or elute at higher salt levels than the full mature virus particles. The benefits of the CEX step were also demonstrated at scale where it delivered a concentrated product with high yields and free of empty procapsids and impurities.

### Example 5: Amplification and Purification of Enteroviruses with GSH Affinity Chromatography

To demonstrate the wide applicability of GSH affinity purification for enteroviruses, 8 different serotypes, encompassing several enterovirus species including Enterovirus B, Enterovirus C, Rhinovirus A, and Rhinovirus B, were evaluated. The strains were purchased from the American Type Culture Collection (ATCC) and amplified in two infections using two cell lines and upstream conditions (Table 4) based on infection protocols commonly used for producing enteroviruses. Cells were planted in tissue culture-treated vented flasks in growth media. Several days post plant, the growth media were decanted and 1 mL of enterovirus inoculum was added to the cell layer. The flasks were incubated for 2 hours before 39 mL of production media were added to each flask and incubated based on the upstream condition used. Upon confirmation of cytopathic effect, the flasks were harvested by collecting the supernatant. The harvests were then stored at −70° C. until they were purified via GSH affinity chromatography.

TABLE 4
Enterovirus species and serotypes, and their production conditions,
purified via GSH affinity chromatography

| | Enterovirus | Production | Upstream |
|---|---|---|---|
| Enterovirus Serotype | Species | Cell Line | Condition |
| Echovirus 1 | Enterovirus B | A | A |
| Rhinovirus 1B | Rhinovirus A | B | D |
| Rhinovirus 35 | Rhinovirus B | B | D |
| Coxsackievirus A 13 | Enterovirus C | A | A |
| Coxsackievirus A 15 | Enterovirus C | A | A |
| Coxsackievirus A 18 | Enterovirus C | A | A |
| Coxsackievirus A 20b | Enterovirus C | B | A |
| Coxsackievirus A 21 | Enterovirus C | B | A |
| Coxsackievirus A 21 | Enterovirus C | B | D |

GSH affinity chromatography was performed using RoboColumns packed with 0.6 mL of Glutathione Sepharose® 4 Fast Flow resin, (GSH Sepharose 4 FF from Cytiva Life Sciences). For each purification, the columns were equilibrated with 5 CVs of Phosphate Buffered Saline (PBS), pH 7.4. Following equilibration, the columns were loaded with 50 CVs of thawed and clarified harvest. Post loading, the columns were washed sequentially with 5 CVs of wash 1 buffer (15 mM Tris, 400 mM NaCl, 1 mM DTT, 0.005% w/v PS-80, pH 8.0) and 5 CVs of wash 2 buffer (15 mM Tris, 150 mM NaCl, 1 mM DTT, 0.005% w/v PS-80, pH 8.0). The columns were eluted with 5 CVs of elution buffer (15 mM Tris, 150 mM NaCl, 1 mM DTT, 1 mM GSH, 0.005% w/v PS-80, pH 8.0) and stripped with 5 CVs of a buffer containing 15 mM Tris, 1000 mM NaCl, 1 mM DTT, 10 mM GSH, 0.005% w/v PS-80, pH 8.0. All steps were performed with a residence time of 4 min and fractions were collected every 200 μL in UV plates (Corning Inc.).

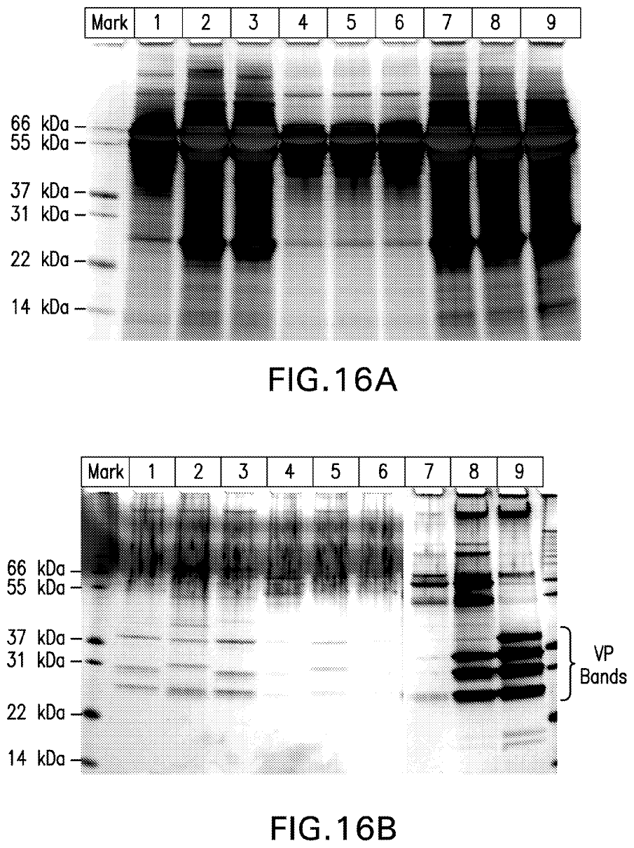

Chromatograms were generated by measuring the optical absorbance of fractions at 260 nm and 280 nm. An elution peak, corresponding typically to a single fraction, was observed between 60-65 CVs. The clarified harvest (FIG. 16A) and elution fractions (FIG. 16B) for each purification were assayed by SDS-PAGE. The gel images showed that GSH affinity chromatography can be used effectively to purify multiple serotypes of enterovirus across a range of different species since enterovirus capsid viral proteins were detectable in the elution product for all tested serotypes while the vast majority of impurities present in the harvest were absent.

### Example 6: GSH Affinity Chromatography Purification of CVA21 Using Clarified Harvests Produced with Different Upstream Conditions

GSH affinity chromatography was evaluated across 2 experiments with CVA21 clarified harvests produced using upstream cell culture conditions A-C (Table 5). 20 mL HiPrep columns packed with GSH Sepharose 4 Fast Flow resin were used on an Äkta Pure 150M FPLC system with UNICORN™ system control software (Cytiva Life Sciences). The CVA21 clarified cell culture harvests were loaded to the column at a flow rate of 100 cm hr⁻¹ until a column loading of 200 CVs was reached. The GSH column was washed at a flow rate of 150 cm hr⁻¹ with 8 CVs of a GSH Wash 1 buffer containing 15 mM Tris, 400 mM NaCl, 0.005% w/v PS-80, pH 8.0 and then 4 CVs of a GSH Wash 2 buffer containing 15 mM Tris, 75 mM NaCl, 0.005% w/v PS-80, 1 mM DTT, pH 8.0. The bound CVA21 particles were eluted with 4 CVs of a GSH Elution solution containing 15 mM Tris, 75 mM NaCl, 0.005% w/v PS-80, 1 mM DTT, 1 mM GSH, pH 8.0 at a flow rate of 150 cm hr⁻¹. The GSH column was stripped with 4 CVs of a GSH Strip buffer containing 15 mM Tris, 1000 mM NaCl, 0.005% w/v PS-80, 1 mM DTT, 10 mM GSH, pH 8.0 and regenerated with a 0.1 N NaOH, 1 M NaCl solution at a flow rate of 150 cm hr⁻¹.

TABLE 5
Experiments employed in the evaluation of the purification
of CVA21 clarified harvests via GSH affinity chromatography

| Experiment | Experiment A | | Experiment B | | |
|---|---|---|---|---|---|
| Experiment | 1 | 2 | 3 | 4 | 5 |
| Arm | | | | | |
| Upstream | B | A | B | C | A |
| Condition | | | | | |

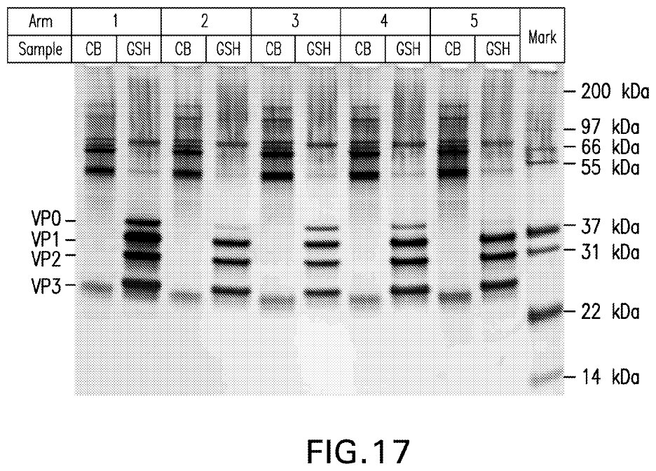

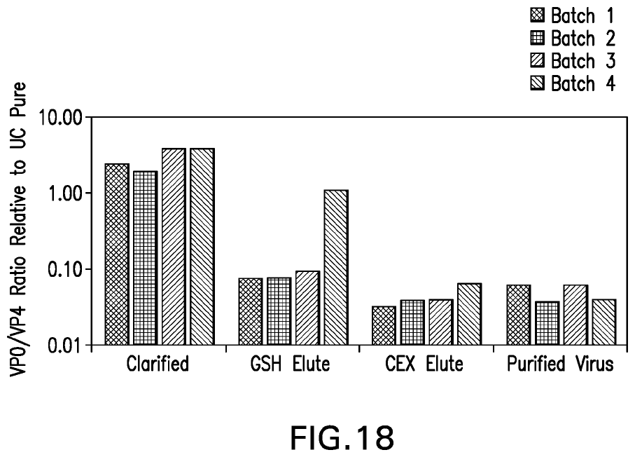

The clarified harvest and GSH elution product samples were analyzed by SDS-PAGE with silver stain (FIG. 17) and capillary electrophoresis anti-VP4 western to detect VP0:VP4 ratio relative to an ultracentrifugation purified virus (FIG. 18). Analysis of SDS-PAGE showed the impurity protein clearance in the GSH elution was similar for all arms, but the GSH elution samples had different intensities of the VP0 band (˜37 kDa) relative to the other viral protein bands. Arm 1, 3, and 4 had higher VP0 content, while Arm 2 and 5 had low VP0 content, indicating differences in empty procapsid clearance across the GSH chromatography step. The empty procapsid:full mature virus particle ratio relative to an ultracentrifugation purified virus by anti-VP4 western demonstrated that the VP0:VP4 ratio decreased from the clarified harvest to the GSH elution product for all arms, but there were differences in the reduction factor. In Arms 2 and 5, which was produced from upstream condition A, the empty procapsid:full mature virion ratio was significantly less than that of ultracentrifugation. These results demonstrate that under some upstream conditions, a fraction of empty procapsids may bind to GSH resin, and a second step such as cation exchange (CEX) chromatography may be implemented to clear the residual empty procapsids in the GSH elution product to meet or exceed the purity of an ultracentrifugation purified virus.

### Example 7: Purification of Enterovirus Using a Process Involving GSH Affinity Chromatography and CEX Chromatography

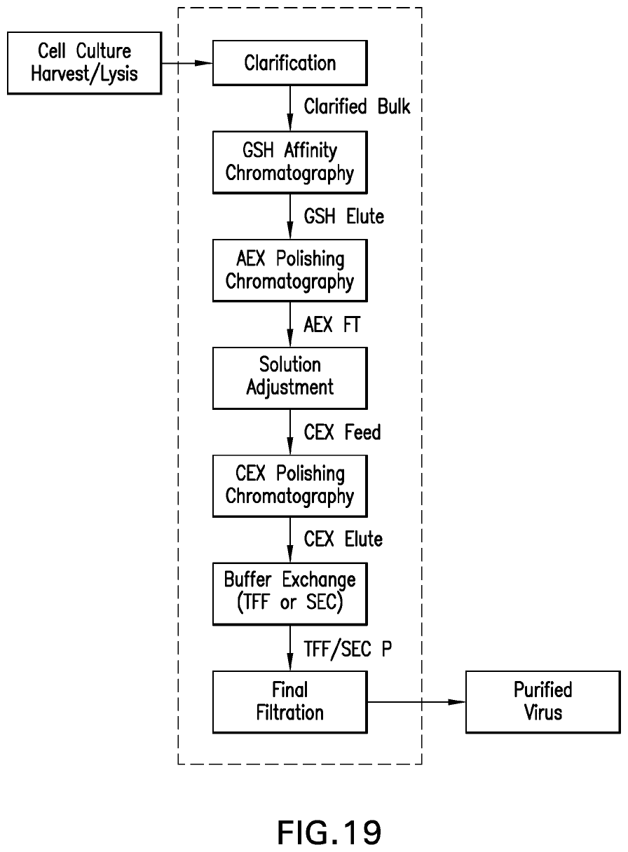

A scalable purification of enteroviruses was demonstrated using the process in FIG. 19 with CVA21 purification from a large-scale bioreactor cell culture harvest as an example. The purification process involves the harvest of enterovirus cell culture consisting of cell culture media, host cell debris, serum impurities, and enterovirus particles through one or multiple clarification filters with a pore size range of 0.2-100 μm to remove host cell debris. A series of two clarification steps may be used with a primary clarification step with a filter pore size of 1-100 μm and a secondary clarification step with a filter pore size of 0.2-5 μm. For harvests from microcarrier cell culture, the primary clarification may involve a mesh bag or a depth filter to remove microcarriers prior to the secondary clarification. In the current example with CVA21, the clarification step was run continuously with 2 filters in series operated at 100 L m⁻² hr⁻¹ (LMH); Primary clarification with a Clarisolve® 60 HX (Merck Millipore, MA, USA) 60 μm depth filter to remove microcarriers and large cell debris, and a secondary clarification with a Sartopure® GF+(Sartorius AG, Göttingen, Germany) 1.2 μm depth filter to clear smaller cell debris including HC-DNA.

In some enterovirus cell cultures, the lytic activity of the virus is sufficient to lyse the cells and no lysis step is needed. In other enterovirus cell cultures, a lysis step such as detergent lysis with PS-80, PS-20, or other surfactant ranging from 0.01-2% w/v may be implemented prior to the clarification step to fully lyse the cells. In the current example with CVA21, no lysis step was performed.

The clarified harvest is loaded directly to a GSH affinity chromatography column. For the GSH chromatography operation, GSH immobilized resin is packed into manufacturing scale chromatography columns and operated with a chromatography skid such as Äkta Pilot or Äkta Ready (both from Cytiva Life Sciences). In the current example with CVA21, a 14 cm diameter column packed with GSH Sepharose 4 FF was used on the Äkta Pilot with UNICORN system control software (Cytiva Life Sciences). The CVA21 clarified cell culture harvest was loaded to the column at a flow rate of 100 cm hr⁻¹ until a column loading of 150-200 CVs. The GSH column was washed at a flow rate of 150 cm hr⁻¹ with 8 CVs of a GSH Wash 1 buffer containing 15 mM Tris, 400 mM NaCl, 0.005% w/v PS-80, pH 8.0 and then 4 CVs of a GSH Wash 2 buffer containing 15 mM Tris, 150 mM NaCl, 0.005% w/v PS-80, 1 mM DTT, pH 8.0. The bound CVA21 particles were eluted with 4 CVs of a GSH Elution solution containing 15 mM Tris, 150 mM NaCl, 0.005% w/v PS-80, 1 mM DTT, 1 mM GSH, pH 8.0 at a flow rate of 150 cm hr⁻¹. The GSH column was stripped with 4 CVs of a GSH Strip buffer containing 15 mM Tris, 1000 mM NaCl, 0.005% w/v PS-80, 1 mM DTT, 10 mM GSH, pH 8.0 and regenerated with a 0.1 N NaOH, 1 M NaCl solution at a flow rate of 150 cm hr⁻¹.

The GSH elution product is loaded directly to an optional polishing anion exchange (AEX) chromatography step operated in flow-through mode for additional residual impurity clearance. The AEX chromatography step may use common AEX chromatography media such as Poros™ 50 HQ (ThermoFisher Scientific), Capto Q (Cytiva Life Sciences), or Nuvia Q (Bio-Rad) or other AEX stationary phases. For large scale AEX chromatography operation, AEX resin is packed into manufacturing scale chromatography columns and run with a chromatography skid such as Äkta Pilot at a flow rate of 50-300 cm hr⁻¹. The AEX column is equilibrated in 3-5 CVs AEX Equilibration buffer composed of a solution at pH 6-9 and a monovalent salt concentration of 50-500 mM. The GSH elution product in a solution at pH 6-9 and a monovalent salt concentration of 50-500 mM is loaded to the AEX column followed by a 1-3 CV chase with the AEX equilibration buffer. The enterovirus particles flow through while impurities including HC-DNA and impurity protein bind to the AEX resin. The column is stripped with 3-5 CVs of AEX Strip buffer composed of a solution at pH 6-9 and a monovalent salt concentration of 500-1500 mM and regenerated with a solution containing 0.1-0.5 N sodium hydroxide. The AEX buffer solutions may contain a surfactant such as PS-80, PS-20 or other similar surfactant at a concentration of 0.001-1% w/v. In the current example with CVA21, a 5 cm diameter column packed with Poros™ 50 HQ resin was run on an Äkta Pilot with UNICORN system control software at a flowrate of 200 cm hr⁻¹. The AEX column was equilibrated with 4 CVs of an AEX equilibration buffer consisting of 15 mM Tris, 150 mM NaCl, 0.005% w/v PS-80, pH 8.0. The GSH elution product containing CVA21 particles was loaded to the column until a loading of 25-CVs and chased with 2 CVs of AEX equilibration buffer. The CVA21 particles flowed through while residual impurities bound to the column. The AEX column was stripped with 4 CVs of an AEX Strip buffer containing 15 mM Tris, 1000 mM NaCl, 0.005% w/v PS-80, pH 8.0 and regenerated with 4 CVs of a 0.5 N NaOH solution. The AEX chromatography step may be omitted if the desired residual impurity specifications in the final purified composition are met without AEX. In this situation, the GSH elution product is forwarded to the solution adjustment step.

In the solution adjustment step, the AEX FT or GSH elution (if AEX is not performed) product is adjusted to solution conditions compatible with binding to the CEX chromatography resin in the subsequent CEX chromatography step. The AEX FT or GSH elution product is initially in a solution at pH 6-9 and a monovalent salt concentration of 50-500 mM. If necessary, concentrated stock solutions of 0.5-1.5 M adjustment buffer solution, consisting of a buffer species such as citrate, at pH 3.5-6.0 and 2-5 M adjustment monovalent salt solution, such as NaCl, are spiked into the AEX FT to bring the solution pH down to pH 3.5-6.0 and increase the monovalent salt concentration to 50-500 mM. One or both adjustment solutions may not be required if the AEX FT is already at the target pH or monovalent salt concentration of the loading solution to the CEX step. In the current example with CVA21, a 1 M sodium citrate, pH 4.0 solution and a 5 M NaCl solution are spiked into the AEX FT, initially at pH 8.0 and 150 mM NaCl, to target a final sodium citrate concentration of 50 mM at pH ˜4.1 and a final NaCl concentration of 400 mM. The concentrated stock solutions were slowly added to the AEX FT product over 5-10 minutes with mixing. This solution adjusted sample was designated CEX feed and represented the target CEX loading solution.

The CEX chromatography step, operated in bind-elute mode, is implemented to improve process robustness as a secondary step for empty procapsid clearance, to clear residual impurities, and to provide additional volume reduction. The CEX step may use common chromatography media such as Poros™ 50 HS (ThermoFisher Scientific), Capto S (Cytiva Life Sciences), or Nuvia S (Bio-Rad) or other CEX stationary phases. For large scale CEX chromatography operation, CEX resin is packed into large scale chromatography columns and run with a chromatography skid such as Äkta Pilot at a flow rate of 50-300 cm hr⁻¹. The CEX column is equilibrated in 3-5 CVs of CEX Equilibration buffer composed of a solution at pH 3.5-6.0 and a monovalent salt concentration of 50-500 mM. The CEX feed in a CEX loading solution at pH 3.5-6.0 and a monovalent salt concentration of 50-500 mM is loaded to the CEX column. The enterovirus particles bind to the CEX resin while some residual impurities may flow through. The CEX column is washed with 3-5 CVs of a CEX Wash buffer solution, composed of a solution at pH 3.5-6.0 and a monovalent salt concentration of 100-600 mM, to remove residual impurities. The full mature virions are selectively eluted from the CEX column using 3-5 CVs of a CEX elution buffer solution, composed of a solution at pH 3.5-4.8 and a monovalent salt concentration of 200-1000 mM NaCl, while the empty procapsids remain bound to the CEX resin. The empty procapsids and other residual impurities are eluted with 3-5 CVs of CEX Strip buffer, composed of a solution at pH 4.0-8.0 and a monovalent salt concentration of 500-1500 mM and the CEX column is regenerated with a solution containing 0.1-0.5 N sodium hydroxide. The CEX buffer solutions may contain a surfactant such as PS-80, PS-20 or other similar surfactant at a concentration of 0.001-1% w/v. In the current example with CVA21, a 5 cm diameter column packed with Poros™ 50 HS resin was run on an Äkta Pilot with UNICORN system control software at a flowrate of 200 cm hr⁻¹. The CEX column was equilibrated with 4 CVs of an CEX equilibration buffer consisting of 50 mM sodium citrate, 400 mM NaCl, 0.005% w/v PS-80, pH 4.0. The CEX feed product containing CVA21 particles was loaded to the column until a loading of 25-30 CVs. The column was washed with 4 CVs of a CEX Wash buffer consisting of 25 mM sodium citrate, 500 mM NaCl, 0.005% w/v PS-80, pH 4.0. The full mature CVA21 virions were selectively eluted from the CEX column with 4 CVs of a CEX elution buffer consisting of 25 mM sodium citrate, 800 mM NaCl, 0.005% w/v PS-80, pH 4.0. The empty CVA21 procapsids were eluted with 4 CVs of a CEX strip buffer consisting of 25 mM sodium citrate, 1000 mM NaCl, 0.005% w/v PS-80, pH 7.0 and the column was regenerated with 4 CVs of a 0.5 N NaOH solution.

The CEX elution product, consisting of purified full mature enterovirus virions, is buffer exchanged into a stabilizing buffer by ultrafiltration/diafiltration (UF/DF) via tangential-flow filtration (TFF) or size-exclusion chromatography (SEC) in desalting mode. For TFF, the enterovirus particles are retained by a hollow fiber or a cassette with a molecular weight cut-off of about 50-500 kDa, while other small solution components permeate through the membrane. The TFF may be operated with a crossflow shear rate of about 1,000-8,000 s⁻¹, a transmembrane pressure (TMP) of about 0.1-10 psig, and a permeate flux of about 5-60 L m⁻² hr⁻¹. The CEX elution product is diafiltered with 5-10 diavolumes into a 1× stabilizing buffer solution consisting of a buffering species at about pH 6-8. A UF step may be performed before or after DF. An optional neutralization step may be performed prior to TFF where the CEX elution product is diluted 2-5-fold into a 2-5× concentrated stabilizing buffer solution. An optional filtration step consisting of a filter with a pore size of about 0.1-1 μm may be used prior to TFF. For buffer exchange with SEC, the CEX elution product is loaded to SEC column packed with resin such as Sephadex (Cytiva Life Sciences) and operated in desalting mode using a chromatography skid such as Äkta Pilot. In the current example with CVA21, the CEX elution product was neutralized by diluting 3-fold into a 3× concentrated stabilizing buffer solution. The neutralized CEX elution product was filtered using a Durapore® 0.22 μm filter (Merck Millipore) to generate a TFF feed solution. The TFF feed solution was initially concentrated 2-3-fold and then buffer exchanged into the 1× stabilizing buffer solution using a Spectrum 300 kDa hollow fiber filter (Repligen) at a crossflow of 2000 s⁻¹, TMP of 1-2 psig, and permeate flux of 20-40 LMH.

A final filtration step is performed with the buffer exchanged TFF or SEC elution product. A filter pore size of 0.1-0.5 μm is used. The final purified composition of enterovirus in the stabilizing buffer solution is frozen and stored at <−60° C. In the current example with CVA21, a Durapore 0.22 μm filter (Merck Millipore) was used.

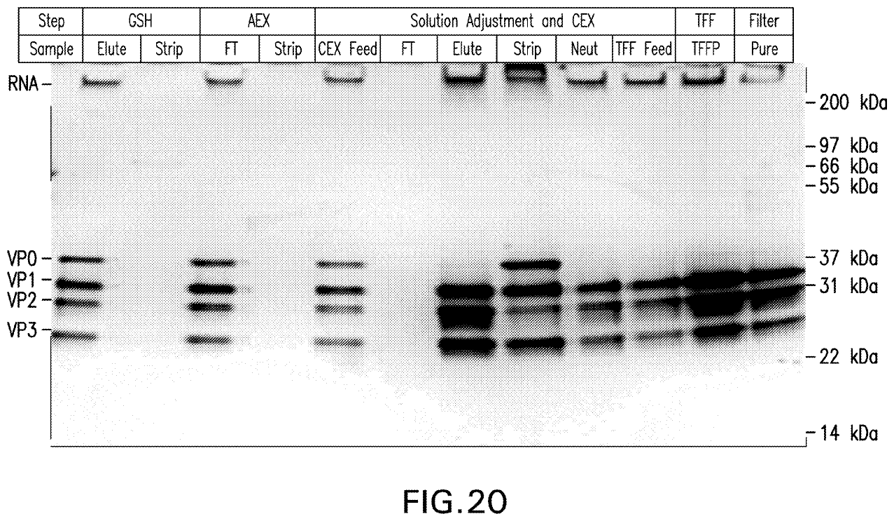

The CVA21 purification process detailed above was demonstrated for 4 batches produced from upstream cell culture conditions A and B. As an example, the purification process intermediate samples for Batch 4 with cell culture condition B were characterized by SDS-PAGE with silver stain (FIG. 20). The GSH elution product demonstrated high purification of residual protein impurities with only VP0, VP1, VP2, VP3 (VP4, 7 kDa, ran off gel), and RNA detectable bands and with high yields (Table 5). The combination of VP0 and VP2 content indicated the GSH elution product contained a distribution of empty procapsid and mature virions. Trace amounts of residual impurities were cleared in the AEX Strip and CEX FT. The CEX elution product had a high concentration of only VP1, VP2, VP3, and RNA bands visible, confirming the clearance of empty procapsids and a pure composition of full mature virions. Similar to the GSH step, high yields were also observed for the CEX elution product (Table 5). The empty capsids were eluted in the CEX strip sample, evidenced by the high VP0 content. The VP band distribution remained constant after the CEX elution product was neutralized and filtered prior to the TFF buffer exchange and final filtration steps.

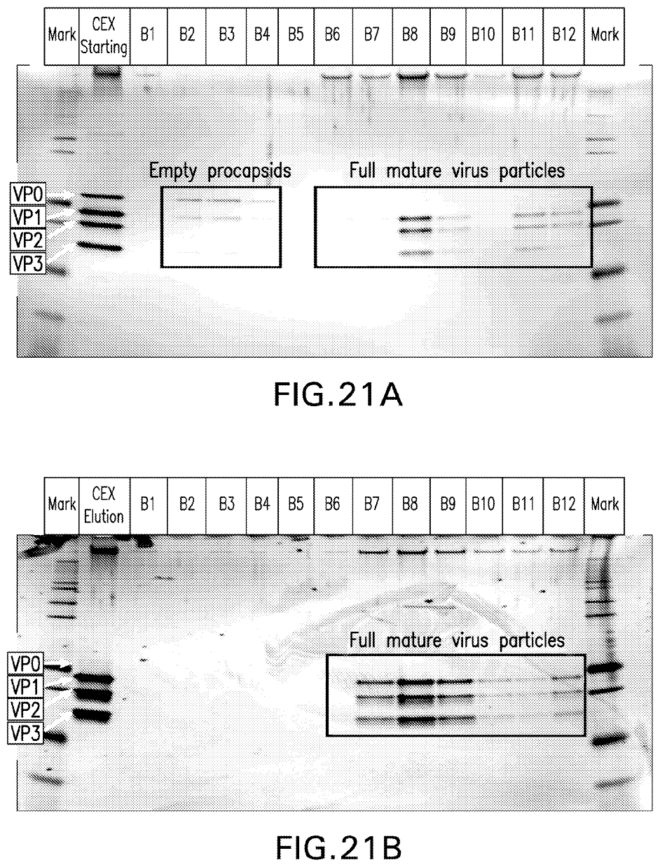

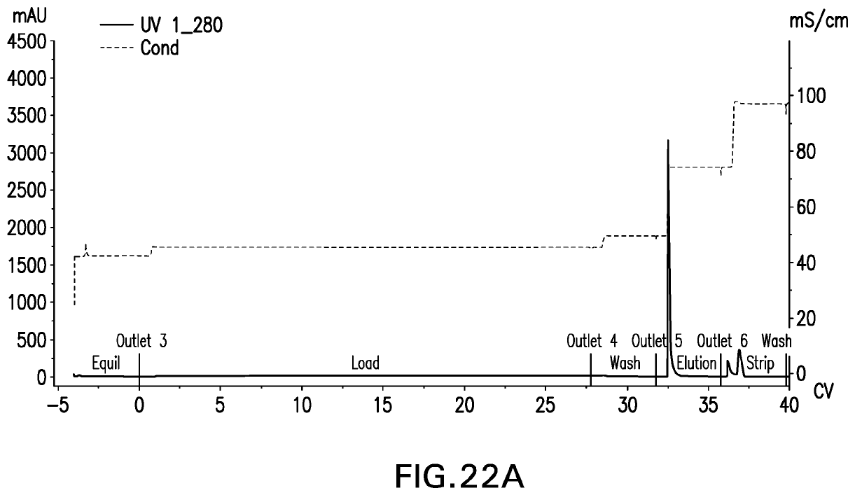

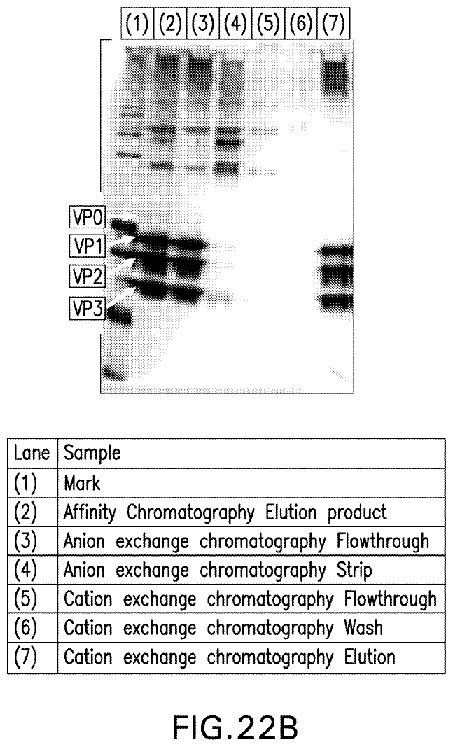

For Batch 4, the starting material was also analyzed through sucrose gradient analysis and it was shown to be rich in both empty procapsids and full mature virus particles (FIG. 21A) while the elution pool was demonstrated to be clear of empty procapsids and of any other type of viral particles (FIG. 21B). The strong elution peak (FIG. 22A) and the gel image of the analyzed fractions (FIG. 21B) demonstrate that the CEX step is not only successful in delivering a pure product, free of empty procapsids, but it is also capable of concentrating it since ˜27 CVs of load are eluted within 4 CVs with excellent yields (Table 6). This is an added benefit to the CEX process running in bind and elute mode since it reduces, for example, the size of the subsequent unit operations and the associated costs. Finally, analysis of concentrated processed intermediates via SDS-PAGE (FIG. 22B), generated from upstream condition A, shows that the CEX step can indeed flow through trace amounts of HCPs that are not cleared through the first two steps in the 3-column purification process (FIG. 19). Hence, the CEX step is scalable and maintains its excellent performance at large scale.

TABLE 6
Step yields based on three analytical methods from processing
Batch 4 via the purification process

| Batch 4 | Plaque | RT-qPCR | HPSEC |
|---|---|---|---|
| Step Yields | Infectivity | Genomes | Particles |
| Clarified Harvest to GSH | 85% | 84% | n/a |
| Elute Step Yield | | | |
| GSH Elute to CEX Elute | 83% | 87% | 77% |
| Step Yield | | | |

### Example 8: Capture and Purification of Alternative Enteroviruses Via Cation Exchange Chromatography Bind and Elute Mode

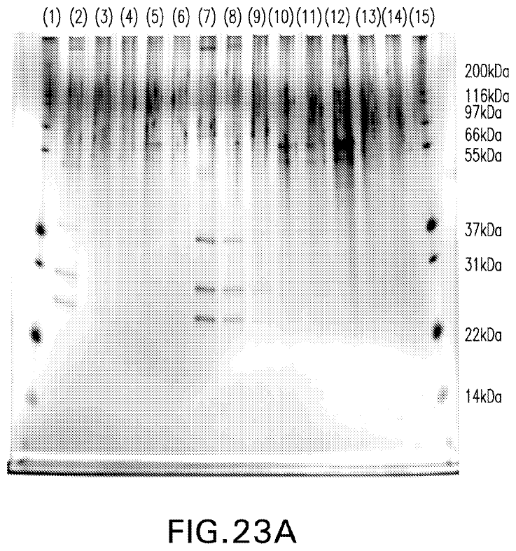

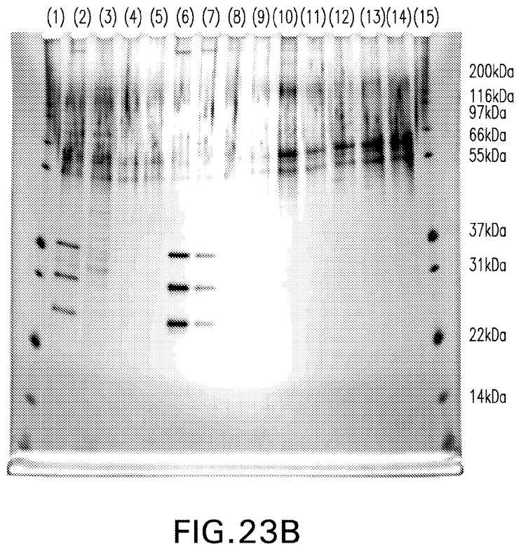

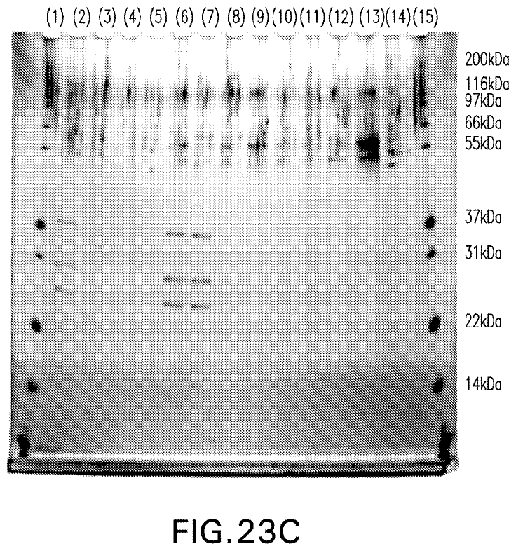

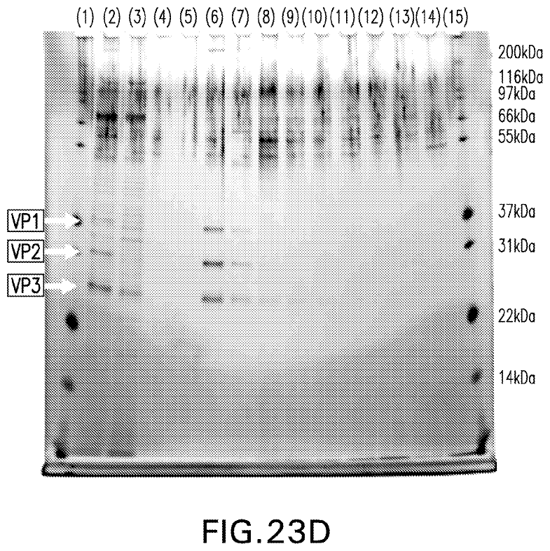

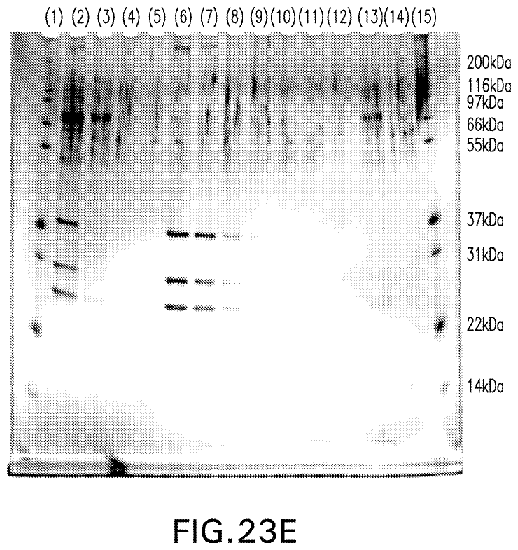

The observations that multiple cation exchange resins resulted in a good separation between full mature virus particles and empty procapsids, along with the fact that residual HCPs could flow through while virus particles bound to the resins, led to the exploration of cation exchange chromatography as a purification step for additional enteroviruses to CVA21. Five enterovirus serotypes were tested to support this: (1) Coxsackievirus A13 (CVA13), (2) Coxsackievirus A15 (CVA15), (3) Coxsackievirus A18 (CVA18), (4) Human Rhinovirus 1B (RV1B), and (5) Human Rhinovirus 35 (RV35). For these tests, enterovirus stocks were purified using small scale columns packed with 200 μL of affinity chromatography resin and the elution products were adjusted to a pH of 4.0 and a salt level of 100 mM NaCl. These were then further purified using 96 well plate batch chromatography as described in Table 7. Here, the plates were pre-dispensed with 20 μL of resin Capto™ S ImpAct (Cytiva, MA, USA) since this resin was also found to provide good purification for CVA21 (FIGS. 5C and 22A). The fractions collected from the batch experiment were analyzed via SDS-PAGE (FIG. 23). For this analysis the gels were developed for a longer period of time than usual due to the low starting protein concentration. The recorded gel images showed three prominent bands in the load, believed to be viral proteins, and which were concentrated in the elution fractions. Hence, for all tested serotypes, the flow through fractions showed zero to low content in viral proteins. Instead, as observed in the case of CVA21 (FIG. 13C), the flow through contained non-binding HCP impurities, with the exception of CVA13 (FIG. 23A) for which the starting protein load was too low to observe bands even in overdeveloped gels for the flowthrough fraction. As a result, the elution fractions showed a concentration and purification effect with the majority of viral proteins eluted in the fractions corresponding to salt levels of 400 mM and 550 mM NaCl. The fact that this behavior was observed for additional enterovirus serotypes to CVA21 demonstrates the wide applicability of cation exchange chromatography as a polishing step for enteroviruses.

TABLE 7
Details of batch chromatography experiments using 96 well PreDictor ™ plates pre-dispensed
with 20 μL of Capto ™ S ImpAct. The plates were handled in a total of 12 steps from which
fractions were collected and analyzed for the last 11 steps. At each step a given liquid volume was
loaded to each well of the chromatography plate with a different composition. Each step was repeated
a number of cycles with an incubation period in between during which plates were shaken. The plates
were evacuated for fraction and effluent collection via centrifugation at 500 g for 5 min periods.

| | | | | Mobile | |
|---|---|---|---|---|---|
| | | | | phase or | |
| | | | Incubation | Load | |
| | | Number | Duration | Volume | |
| a/a | Step | of Cycles | (min) | (μL) | Mobile phase Composition or Load |
| 1 | Equilibration | 3 | 5 | 300 | 50 mM Citrate, 100 mM NaCl, 0.005% PS80, |
| | | | | | pH 4.0 |
| 2 | Load | 2 | 60 | 300 | Affinity Chromatography elution product, |
| | | | | | adjusted to 100 mM NaCl and pH 4.0 |
| 3 | Wash | 1 | 5 | 100 | 50 mM Citrate, 100 mM NaCl, 0.005% PS80, |
| | | | | | pH 4.0 |
| 4 | Elution 1 | 1 | 5 | 100 | 50 mM Citrate, 250 mM NaCl, 0.005% PS80, |
| | | | | | pH 4.0 |
| 5 | Elution 2 | 1 | 5 | 100 | 50 mM Citrate, 400 mM NaCl, 0.005% PS80, |
| | | | | | pH 4.0 |
| 6 | Elution 3 | 1 | 5 | 100 | 50 mM Citrate, 550 mM NaCl, 0.005% PS80, |
| | | | | | pH 4.0 |
| 7 | Elution 4 | 1 | 5 | 100 | 50 mM Citrate, 700 mM NaCl, 0.005% PS80, |
| | | | | | pH 4.0 |
| 8 | Elution 5 | 1 | 5 | 100 | 50 mM Citrate, 850 mM NaCl, 0.005% PS80, |
| | | | | | pH 4.0 |
| 9 | Elution 6 | 1 | 5 | 100 | 50 mM Citrate, 1000 mM NaCl, 0.005% PS80, |
| | | | | | pH 4.0 |
| 10 | Elution 7 | 1 | 5 | 100 | 50 mM Citrate, 1150 mM NaCl, 0.005% PS80, |
| | | | | | pH 4.0 |
| 11 | Elution 8 | 1 | 5 | 100 | 50 mM Citrate, 1300 mM NaCl, 0.005% PS80, |
| | | | | | pH 4.0 |
| 12 | Strip | 2 | 5 | 100 | 100 mM Tris, 1000mM NaCl, 0.005% PS80, |
| | | | | | pH 8.0 |

### REFERENCES

1. Baggen J, Thibaut H J, Strating J R P M, van Kuppeveld F J M. (2018) The life cycle of non-polio enteroviruses and how to target it. Nature Reviews Microbiology, 16, 368-381.
2. Yin E J, Shin Y J, Kim J H, Kim T G, Chang S Y. (2017) Enterovirus 71 infection and vaccines. Clinical and Experimental Vaccine Research, 6, 4-14.
3. Pons-Salot M, Parker E P K, Grassly N C. (2015) The epidemiology of non-polio enteroviruses. Current Opinion of Infectious Diseases, 28, 479-487.
4. Kaufman H L, Kohlhapp F J, Zloza A. (2015) Oncolytic viruses: a new class of immunotherapy drugs. Nature Reviews Cancer Immunotherapy, 14, 642-662.
5. Bradley S, Jakes A D, Harrington K, Pandha H, Melcher A, Erringon-Mais F. (2014). Applications of coxsackievirus A21 in oncology. Oncolytic Virotherapy, 3, 47-55.
6. Shingler K L, Organtini L J, Hafenstein S. (2016). Enterovirus 71 virus propagation and purification. Bio Protocols, 4(9), e1117.
7. Tomono T, Hirai Y, Okada H, Adachie K, Ishii A, Shimada T, Onodera M, Tamaoka A, Okada T. (2016) Ultracentrifugation-free chromatography-mediated large-scale purification of recombinant adeno-associated virus serotype 1 (rAAV1). Molecular Therapy. Methods & Clinical Development, 3, 15058.
8. Jiang P, Liu Y, Ma H C, Paul A V, Wimmer E. (2014) Picornavirus Morphogenesis. Microbiology and Molecular Biology Reviews, 78(3), 418-437.
9. Lennette et al., 1958 (Am. J. Hyg. 68: 272)
10. Jansson, J. C.; Rydén, L. Protein Purification; Wiley: New York, 1998
11. Pompella A, Visvikis A, Paolicchi D, De Tata V, Casini A F. (2003). The changing faces of glutathione, a cellular protagonist. Biochemical Pharmacology, 66, 1499-1503.
12. Stenzel M H, ACS Macro Letters, 2, 14-18 (2013)
13. Dalldorf et al., 1949 (J. exp. Med. 89: 567)
14. Sickles et al., 1959 (Proc. Soc. exp. Biol. Med. 102: 742-743)
15. Abraham and Cheever, 1963. (PSEBM 112: 981)
16. Mirkovic et al., 1974 (Intervirol. 4: 119-127)
17. Konstantinidis, S., Goh, H. Y., Martin Bufájer, J. M., de Galbert, P., Parau, M., & Velayudhan, A. (2018). Flexible and accessible automated operation of miniature chromatography columns on a liquid handling station, Biotechnology Journal, 13(3), 1700390.
18. Loughney, J. W., Lancaster, C., Ha, S., & Rustandi, R. R. (2014). Residual bovine serum albumin (BSA) quantitation in vaccines using automated Capillary Western technology. Analytical biochemistry, 461, 49-56.

U.S. provisional application No. 63/126,743, filed Dec. 17, 2020, and U.S. provisional application No. 63/211,162, filed Jun. 16, 2020 are incorporated by reference in their entirety. All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents. To the extent that the references provide a definition for a claimed term that conflicts with the definitions provided in the instant specification, the definitions provided in the instant specification shall be used to interpret the claimed invention.

1. A method of purifying an enterovirus comprising the steps of:
a. binding the enterovirus to a cation exchange stationary phase using a loading solution with a pH of about 3.5 to 6.0;
b. eluting the enterovirus from the stationary phase with an elution solution with a pH of about 3.5 to 4.8.

2. The method of claim 1, wherein prior to step (a), the stationary phase is equilibrated with an equilibration solution.

3. The method of claim 1, further comprising step (i) of washing the stationary phase with one or more wash solutions after step (a) but prior to step (b).

4. The method of claim 3, wherein step (i) comprises a wash step with a wash solution having a conductivity higher than the equilibration solution or loading solution.

5. The method of claim 1, wherein one or more of the loading solution, equilibration solution, the one or more wash solutions and the elution solution has a pH of about 3.8-4.5.

6. The method of claim 1, wherein the elution solution has a pH of about 3.8-4.5.

7. The method of claim 1, wherein one or more of the loading solution, equilibration solution, the one or more wash solutions and the elution solution has a pH of about 4.0.

8. The method of claim 1, wherein one or more of the loading solution, equilibration solution, the one or more wash solutions and the elution solution further comprises a surfactant.

9. The method of claim 8, wherein the surfactant is PS-80 or PS-20.

10. The method of claim 8, wherein the surfactant is about 0.001-1% w/v PS-80.

11. The method of claim 8, wherein the surfactant is about 0.005% w/v PS-80.

12. The method of claim 1, wherein the loading solution or equilibration solution comprises about 50-500 mM monovalent salt.

13. The method of claim 12, wherein the loading solution or equilibration solution comprises up to about 350 mM monovalent salt.

14. The method of claim 3, wherein the one or more wash solutions comprises about 50-600 mM monovalent salt.

15. The method of claim 3, wherein the one or more wash solutions comprises about 400-600 mM NaCl or KCl.

16. The method of claim 1, wherein the elution solution comprises about 350-1200 mM of monovalent salt.

17. The method of claim 1, wherein the elution solution comprises about 200-1000 mM NaCl or KCl.

18. The method of claim 1, wherein the elution solution comprises about 550-850 mM NaCl or KCl.

19. The method of claim 1, wherein the cation exchange stationary phase is Poros™ 50 HS.

20. A method of purifying an enterovirus comprising the steps of:
a. applying a loading solution comprising the enterovirus to a cation exchange stationary phase using a loading solution with a pH of about 3.5 to 4.7;
b. collecting the flow-through comprising the enterovirus.

21-50. (canceled)
